(19) United States
(12) Patent Application Publication
(10) Pub. No.: US 2014/0322147 A1
(43) Pub. Date: Oct. 30, 2014

(54) ANTIMICROBIAL COMPOSITION

(71) Applicant: The Trustees of Columbia University in the City of New York, New York, NY (US)

(72) Inventors: Shanta M. Modak, River Edge, NJ (US); Santoshkumar Dongre, Aurangabad (IN)

(21) Appl. No.: 14/323,843

(22) Filed: Jul. 3, 2014

Related U.S. Application Data
(63) Continuation of application No. PCT/US12/71451, filed on Dec. 21, 2012.
(60) Provisional application No. 61/583,482, filed on Jan. 5, 2012.
(60) Provisional application No. 61/583,998, filed on Jan. 6, 2012.
(60) Provisional application No. 61/691,912, filed on Aug. 22, 2012.

(51) Int. Cl. A01N 65/36; A23L 2/44; A61Q 19/00; A61Q 11/00; A61K 8/34; A01N 31/04; A23L 3/3463; A61K 8/97

(52) U.S. Cl. CPC A01N 65/36; A23L 3/34635; A23L 2/44; A61K 8/97; A61Q 11/00; A61K 8/34; A01N 31/04; A61Q 19/00; A61Q 19/007
USPC 424/58; 426/532; 426/330.3; 426/335; 424/736

(57) ABSTRACT

Disclosed herein are antimicrobial compositions and methods of producing an antimicrobial effect in which a citrus extract such as Biosecur® is used in combination with benzyl alcohol and one or more additional agent selected from the group consisting of lauroyl arginate/glyceryl laurate and essential oils or constituents thereof such as galangal oil, thyme oil, thymol, cinnamon leaf oil, cinnamon bark oil, lemongrass oil, orange oil, pine oil, cedarwood oil, curry leaf oil, and rosemary oil. These compositions may be used as natural preservatives for personal care products, foods, beverages, and as topical or surface disinfectants.

### CROSS-REFERENCE TO RELATED APPLICATIONS

This application is a continuation of International Application No. PCT/US12/071,451, filed Dec. 21, 2012, which claims the benefit of and priority to U.S. Provisional Application Ser. No. 61/583,482, filed Jan. 5, 2012, U.S. Provisional Application Ser. No. 61/583,998, filed Jan. 6, 2012, and U.S. Provisional Application Ser. No. 61/691,912, filed Aug. 22, 2012; each of which is hereby incorporated by reference in its entirety, and to each of which priority is claimed.

### 1. INTRODUCTION

Disclosed herein are compositions having antimicrobial activity comprising benzyl alcohol and a citrus extract such as Biosecur,® ethyl lauroyl arginate (or glyceryl laurate) and/or essential oils. These compositions can be used as preservatives or to otherwise impart antimicrobial activity into personal care, food, and household cleaning products.

### 2. BACKGROUND

Antimicrobial agents are widely used in medical supplies, household cleaning, and personal care products. Adverse effects of antimicrobials in current circulation include skin, eye, and lung irritation. Older formulations are under scrutiny due to environmental and health issues. New formulations that address and correct these issues are highly desirable.

Biosecur® is the first certified organic ingredient for use as a broad-spectrum preservative and disinfectant. Biosecur® is derived from 100% organic citrus fruit, but does not contain grapefruit. It has been reported that, in tests performed according to U.S. Food and Drug Administration's (FDA's) Good Laboratory Practice (GLP) protocol, Biosecur® was found to kill more than 99.999% of common bacteria within 15- to 30-seconds after contact. Furthermore, Biosecur® is reported to provide broad-spectrum antimicrobial efficacy against various bacteria, fungi and yeast.

### 3. SUMMARY

Disclosed herein are antimicrobial compositions and methods of producing an antimicrobial effect in which a citrus extract such as Biosecur® is used in combination with benzyl alcohol and one or more additional agent selected from the group consisting of lauroyl arginate (or glyceryl laurate) and essential oils or constituents thereof such as galangal oil, thyme oil, thymol, cinnamon oil (for example, cinnamon leaf oil and/or cinnamon bark oil), lemongrass oil, orange oil, pine oil, cedarwood oil, curry leaf oil, and rosemary oil. These compositions may be used as natural preservatives for personal care products, foods, beverages, and as topical or surface disinfectants.

In the embodiments disclosed in this Summary and the Detailed Description, Biosecur® may optionally be substituted with a citrus extract, meaning an extract of juice, or pulp, or rind, or a combination thereof, derived from a citrus fruit or fruits such as orange, lemon and lime, (in certain non-limiting embodiments, not grapefruit and in other non-limiting embodiments, optionally grapefruit) which comprises at least 0.1 percent citric acid and at least 0.2 percent (1 mg/ml) ascorbic acid. In certain embodiments, the citrus extract comprises one or more citrus bioflavonoids.

“Lauroyl arginate” refers to ethyl lauroyl arginate (e.g., HCl) which may optionally be provided as a 10 percent or 20 percent solution which may further comprise glycerin. In the embodiments disclosed in this Summary and the Detailed Description, ethyl lauroyl arginate may optionally be substituted with equivalent weight amounts of glycerol monolaurate (also known as glyceryl laurate) or propylene glycol monolaurate. “Ethyl lauroyl arginate/glyceryl laurate” means ethyl lauroyl arginate or glyceryl laurate separately or in combination.

In embodiments disclosed in this Summary and the Detailed Description, thymol may optionally be substituted with thyme oil and vice versa, where two or three-fold greater volumes of thyme oil relative to thymol are used.

In the formulations disclosed herein, where the weight percentages of ingredients listed do not add up to 100 percent, the additional components may include components known in the art for an intended use, for example in personal care products, foods, beverages, and as topical or surface disinfectants. As non-limiting examples, the components may comprise one or more of water or other solvent, thickening agent(s), emollient(s), food product, etc., as the case may be.

In certain embodiments, compositions disclosed herein comprise benzyl alcohol (0.001-2.0 percent or 0.3-2.0 percent weight/weight (w/w)), Biosecur® or other citrus extract (0.001-1.0 percent or 0.3-1.0 percent w/w), and one or more of ethyl lauroyl arginate (0.0001-0.5 percent, 0.02-0.5 percent, 0.001-0.015 percent, or 0.5-2 percent w/w), thymol (0.0002-0.1 or 0.02-0.1 percent w/w), galangal oil (0.0002-0.15 or 0.02-0.15 percent w/w) and optionally cinnamon oil (for example, cinnamon bark oil and/or cinnamon leaf oil) (0.0004-0.2 percent, 0.0004-0.1 percent or 0.05-0.2 percent w/w), lemongrass oil (0.02-0.5 percent w/w), orange oil (0.1-0.2 percent w/w), rosemary oil (0.005-0.2 percent w/w), curry leaf oil (0.005-0.5 percent w/w), cedarwood oil (0.2-0.5 percent w/w), pine oil (0.1-0.5 percent w/w), aliphatic alcohol (0.1-20 percent w/w), phenoxy ethanol (0.3-1.0 percent w/w) and/or phenyl ethanol (0.3-1.0 percent w/w).

In certain embodiments, compositions disclosed herein comprise Biosecur® or other citrus extract (0.3-1.0 percent w/w), and one or more of ethyl lauroyl arginate and/or glyceryl laurate (0.05-0.2 percent w/w), thymol (0.02-0.1 percent w/w), cinnamon oil (for example, cinnamon bark oil and/or cinnamon leaf oil) (0.05-1 percent w/w), galangal oil (0.02-0.1 percent w/w), lemongrass oil (0.05-1 percent w/w), and one or more solvents selected from the group consisting of aliphatic alcohol (0.1-20 percent w/w), benzyl alcohol (0.2-1.0 percent w/w), phenoxy ethanol (0.1-1.0 percent w/w), phenyl ethanol (0.1-1.0 percent w/w), glycerin (0.5-3.0 percent w/w), propylene glycol (0.5-3.0% w/w), diglycerol (0.5-3.0 percent w/w), polyglycerol (0.5-3.0 percent w/w), dipropylene glycol (0.5-3.0 percent w/w) and mixtures thereof.

One non-limiting embodiment is a composition, for example a preservative for addition to a personal care product, comprising benzyl alcohol, ethanol, phenyl ethanol or combinations thereof at a concentration of between about 40 and 80 percent (weight/weight), Biosecur® or other citrus extract at a concentration of between about 40 and 80 percent (weight/weight) and one or more agent selected from the group consisting of ethyl lauroyl arginate at a concentration of between about 0.5 and 2 percent (weight/weight), thymol at a concentration of between about 3 and 5 percent (weight/weight), galangal oil at a concentration of between about 3 and 10 percent or between about 3 and 5 percent (weight/weight), cinnamon oil (for example, cinnamon bark oil and/or cinnamon leaf oil) at a concentration of between about 4 and 10 percent (weight/weight) and an organic acid selected from the group consisting of benzoic acid, lactic acid and combinations thereof at a concentration of between about 0 and 20 percent (weight/weight).

One non-limiting embodiment is a composition, for example a personal care product, comprising benzyl alcohol, ethanol, phenyl ethanol or combinations thereof at a concentration of about 31.84 percent (weight/weight), Biosecur® or other citrus extract at a concentration of about 63.69 percent (weight/weight), ethyl lauroyl arginate at a concentration of about 1.27 percent (weight/weight) and thymol at a concentration of about 3.2 percent (weight/weight).

One non-limiting embodiment is a composition, for example a personal care product, comprising benzyl alcohol at a concentration of between about 0.3-1.0 percent (weight/weight), Biosecur® or other citrus extract at a concentration between about 0.3-1.0 percent (weight/weight), and one or more agent selected from the group consisting of ethyl lauroyl arginate at a concentration of between about 0.02-0.05 percent (weight/weight), thymol at a concentration of between about 0.02-0.1 percent (weight/weight), galangal oil at a concentration of between about 0.02-0.15 percent (weight/weight), cinnamon oil (for example, cinnamon bark oil and/or cinnamon leaf oil) at a concentration of between about 0.0004 and 0.1 percent or between about 0.1 and 0.2 percent (weight/weight) and an organic acid selected from the group consisting of benzoic acid, lactic acid and combinations thereof at a concentration of between about 0 and 0.2 percent (weight/weight).

One non-limiting embodiment is a composition, for example a personal care product, comprising benzyl alcohol at a concentration of about 0.5 percent (weight/weight), Biosecur® or other citrus extract at a concentration of about 1.0 percent (weight/weight), ethyl lauroyl arginate at a concentration of about 0.02 percent (weight/weight) and thymol at a concentration of about 0.05 percent (weight/weight).

One non-limiting embodiment is a composition, for example a personal care product, comprising benzyl alcohol at a concentration of between about 10 and 70 percent (weight/weight), Biosecur® or other citrus extract at a concentration of between about 1.0 and 80 percent (weight/weight) and one or more agent selected from the group consisting of thymol at a concentration of between about 0.5 and 20 percent (weight/weight) and an organic acid selected from the group consisting of benzoic acid, lactic acid and combinations thereof at a concentration of between about 0 and 20 percent (weight/weight).

One non-limiting embodiment is a composition, for example a preservative for addition to a food or beverage product, comprising benzyl alcohol at a concentration of between about 10 and 70 percent (weight/weight), Biosecur® or other citrus extract at a concentration of between about 20 and 30 percent (weight/weight) and one or more agent selected from the group consisting of ethyl lauroyl arginate at a concentration of between about 1 and 2 percent (weight/weight), thymol at a concentration of between about 4 and 10 percent (weight/weight), cinnamon oil (for example, cinnamon bark oil and/or cinnamon leaf oil) at a concentration of between about 4 and 10 percent (weight/weight) and galangal oil at a concentration of between about 4 and 10 percent (weight/weight).

One non-limiting embodiment is a composition, for example a food or beverage product, comprising benzyl alcohol at a concentration of between about 0.005 and 0.05 percent (weight/weight), Biosecur® or other citrus extract at a concentration between about 0.001 and 0.15 percent (weight/weight), and one or more agent selected from the group consisting of ethyl lauroyl arginate at a concentration of between about 0.0001 and 0.02 percent (weight/weight), thymol at a concentration of between about 0.0004 and 0.04 percent or between about 0.0004 and 0.1 percent (weight/weight), galangal oil at a concentration of between about 0.0004 and 0.04 percent or between about 0.02 and 0.15 percent (weight/weight), and cinnamon oil (for example, cinnamon bark oil and/or cinnamon leaf oil) at a concentration of between about 0.0004 and 0.1 percent or between about 0.0004 and 0.04 percent (weight/weight).

One non-limiting embodiment is a composition, for example a food or beverage product, comprising benzyl alcohol at a concentration of between about 0.001 and 0.05 percent (weight/weight), Biosecur® or other citrus extract at a concentration between about 0.0001 and 0.15 percent (weight/weight), and one or more agent selected from the group consisting of ethyl lauroyl arginate at a concentration of between about 0.0001 and 0.02 percent (weight/weight), thymol at a concentration of between about 0.0002 and 0.005 percent (weight/weight), galangal oil at a concentration of between about 0.0002 and 0.005 percent (weight/weight) and cinnamon oil (for example, cinnamon bark oil and/or cinnamon leaf oil) at a concentration of between about 0.0004 and 0.04 percent (weight/weight).

One non-limiting embodiment is a composition, for example a preservative for addition to a food or beverage product, comprising benzyl alcohol at a concentration of between about 5 and 30 percent (weight/weight), Biosecur® or other citrus extract at a concentration of between about 2 and 20 percent (weight/weight) and one or more agent selected from the group consisting of ethyl lauroyl arginate at a concentration of between about 1 and 5 percent (weight/weight), thymol at a concentration of between about 0.1 and 0.4 percent (weight/weight), cinnamon oil (for example, cinnamon bark oil and/or cinnamon leaf oil) at a concentration of between about 2 and 10 percent (weight/weight), solvent (for example, ethanol and/or glycerine) at a concentration of between about 5 and 70 percent (weight/weight), and an organic acid selected from the group consisting of benzoic acid, lactic acid and combinations thereof at a concentration of between about 0 and 20 percent (weight/weight).

One non-limiting embodiment is a composition, for example a preservative for addition to a food or beverage product, comprising benzyl alcohol at a concentration of between about 0.01 and 0.06 percent (weight/weight), Biosecur® or other citrus extract at a concentration of between about 0.004 and 0.04 percent (weight/weight) and one or more agent selected from the group consisting of ethyl lauroyl arginate at a concentration of between about 0.002 and 0.01 percent (weight/weight), thymol at a concentration of between about 0.0002 and 0.0008 percent (weight/weight), cinnamon oil (for example, cinnamon bark oil and/or cinnamon leaf oil) at a concentration of between about 0.004 and 0.02 percent (weight/weight) and solvent (for example, ethanol and/or glycerine) at a concentration of between about 0.01 and 0.15 percent (weight/weight).

One non-limiting embodiment is a composition, for example a preservative for addition to a food or beverage product, comprising benzyl alcohol at a concentration of between about 5 and 30 percent (weight/weight), Biosecur® or other citrus extract at a concentration of between about 2 and 20 percent (weight/weight) and one or more agent selected from the group consisting of ethyl lauroyl arginate at a concentration of between about 1 and 5 percent (weight/weight), thymol at a concentration of between about 0.1 and 0.4 percent (weight/weight), cinnamon oil (for example, cinnamon bark oil and/or cinnamon leaf oil) at a concentration of between about 2 and 10 percent (weight/weight), ethanol at a concentration of between about 5 and 70 percent (weight/weight) and glycerine at a concentration of between about 5 and 40 percent (weight/weight).

One non-limiting embodiment is a composition, for example a preservative for addition to a food or beverage product, comprising benzyl alcohol at a concentration of about 0.04 percent (weight/weight), Biosecur® or other citrus extract at a concentration of about 0.01 percent (weight/weight) and one or more agent selected from the group consisting of ethyl lauroyl arginate at a concentration of about 0.004 percent (weight/weight), thymol at a concentration of about 0.0004 percent (weight/weight), cinnamon oil (for example, cinnamon bark oil and/or cinnamon leaf oil) at a concentration of about 0.01 percent (weight/weight), ethanol at a concentration of about 0.08 percent (weight/weight) and glycerine at a concentration of about 0.055 percent (weight/weight).

One non-limiting embodiment is a composition, for example a preservative for addition to a food or beverage product, comprising benzyl alcohol at a concentration of about 20 percent (weight/weight), Biosecur® or other citrus extract at a concentration of about 5 percent (weight/weight) and one or more agent selected from the group consisting of ethyl lauroyl arginate at a concentration of about 2 percent (weight/weight), thymol at a concentration of about 0.2 percent (weight/weight), cinnamon oil (for example, cinnamon bark oil and/or cinnamon leaf oil) at a concentration of about 5 percent (weight/weight) and ethanol at a concentration of about 60 percent (weight/weight).

One non-limiting embodiment is a composition, for example a preservative for addition to a food or beverage product, comprising benzyl alcohol at a concentration of about 0.04 percent (weight/weight), Biosecur® or other citrus extract at a concentration of about 0.01 percent (weight/weight) and one or more agent selected from the group consisting of ethyl lauroyl arginate at a concentration of about 0.004 percent (weight/weight), thymol at a concentration of about 0.0004 percent (weight/weight), cinnamon oil (for example, cinnamon bark oil and/or cinnamon leaf oil) at a concentration of about 0.01 percent (weight/weight) and ethanol at a concentration of about 0.08 percent (weight/weight).

One non-limiting embodiment is a composition, for example a preservative for addition to a food or beverage product, comprising benzyl alcohol at a concentration of between about 0.5 and 30 percent (weight/weight), Biosecur® or other citrus extract at a concentration of between about 5 and 40 percent (weight/weight) and one or more agent selected from the group consisting of ethyl lauroyl arginate at a concentration of between about 0.2 and 4 percent (weight/weight), thymol at a concentration of between about 0.1 and 5 percent (weight/weight), cinnamon oil (for example, cinnamon bark oil and/or cinnamon leaf oil) at a concentration of between about 4 and 10 percent (weight/weight) and an organic acid selected from the group consisting of benzoic acid, lactic acid and combinations thereof at a concentration of between about 0 and 20 percent (weight/weight).

One non-limiting embodiment is composition, for example a food or beverage product, comprising benzyl alcohol at a concentration of between about 0.1 and 1.0 percent (weight/weight), Biosecur® or other citrus extract at a concentration between about 0.1 and 1.0 percent (weight/weight), and one or more agent selected from the group consisting of ethyl lauroyl arginate at a concentration of between about 0.05 and 0.5 percent (weight/weight), thymol at a concentration of between about 0.01 and 0.05 percent (weight/weight), cinnamon oil (for example, cinnamon bark oil and/or cinnamon leaf oil) at a concentration of between about 0.2 and 1.0 percent (weight/weight) and an organic acid selected from the group consisting of benzoic acid, lactic acid and combinations thereof at a concentration of between about 0 and 0.2 percent (weight/weight).

One non-limiting embodiment is a composition, for example a preservative for addition to a food or beverage product, comprising Biosecur® or other citrus extract at a concentration of between about 5 and 40 percent (weight/weight) and one or more agent selected from the group consisting of ethyl lauroyl arginate at a concentration of between about 0.2 and 4 percent (weight/weight), thymol at a concentration of between about 0.1 and 5 percent (weight/weight), cinnamon oil (for example, cinnamon bark oil and/or cinnamon leaf oil) at a concentration of between about 1 and 20 percent (weight/weight) and an organic acid selected from the group consisting of benzoic acid, lactic acid and combinations thereof at a concentration of between about 0 and 20 percent (weight/weight).

One non-limiting embodiment is composition, for example a food or beverage product, comprising Biosecur® or other citrus extract at a concentration between about 0.1 and 1.0 percent (weight/weight), and one or more agent selected from the group consisting of ethyl lauroyl arginate at a concentration of between about 0.05 and 0.5 percent (weight/weight), thymol at a concentration of between about 0.01 and 0.05 percent (weight/weight), cinnamon oil (for example, cinnamon bark oil and/or cinnamon leaf oil) at a concentration of between about 0.2 and 1.0 percent (weight/weight) and an organic acid selected from the group consisting of benzoic acid, lactic acid and combinations thereof at a concentration of between about 0 and 0.2 percent (weight/weight).

One non-limiting embodiment is a composition, for example a disinfectant composition for use in reducing the number and/or growth of microbes on a food product, comprising benzyl alcohol at a concentration of between about 30 and 80 percent (weight/weight), Biosecur® or other citrus extract at a concentration between about 10 and 50 percent (weight/weight), and one or more agent selected from the group consisting of ethyl lauroyl arginate at a concentration of between about 0.2 and 2 percent or between about 2 and 20 percent (weight/weight), thymol at a concentration of between about 2 and 20 percent (weight/weight), galangal oil at a concentration of between about 2 and 20 percent (weight/weight), rosemary oil at a concentration of between about 1 and 10 percent (weight/weight), cinnamon oil (for example, cinnamon bark oil and/or cinnamon leaf oil) at a concentration of between about 4 and 10 percent (weight/weight) and curry leaf oil at a concentration of between about 1 and 10 percent (weight/weight).

One non-limiting embodiment is a composition, for example a diluted disinfectant composition for use in reducing the number and/or growth of microbes on a food product, comprising benzyl alcohol at a concentration of between about 0.05 and 1 percent or between about 0.5 and 1 percent (weight/weight), Biosecur® or other citrus extract at a concentration between about 0.05 and 1 percent or between about 0.5 and 1 percent (weight/weight), and one or more agent selected from the group consisting of ethyl lauroyl arginate at a concentration of between about 0.001 and 0.05 percent or between about 0.001 and 0.05 percent (weight/weight), thymol at a concentration of between about 0.01 and 0.5 percent or between about 0.001 and 0.5 percent (weight/weight), galangal oil at a concentration of between about 0.01 and 0.5 percent or between about 0.001 and 0.5 percent (weight/weight), rosemary oil at a concentration of between about 0.005 and 0.5 percent (weight/weight), cinnamon oil (for example, cinnamon bark oil and/or cinnamon leaf oil) at a concentration of between about 0.0004 and 0.1 percent or between about 0.05 and 0.2 percent (weight/weight), curry leaf oil at a concentration of between about 0.005 and 0.5 percent (weight/weight), and an organic acid selected from the group consisting of benzoic, lactic acid and combinations thereof at a concentration of between about 0 and 0.2 percent (weight/weight).

One non-limiting embodiment is a composition, for example a topical disinfectant composition for use in reducing the number and/or growth of microbes on the skin of a subject, comprising benzyl alcohol at a concentration of between about 0.5 and 1.0 percent (weight/weight), Biosecur® or other citrus extract at a concentration between about 0.5 and 1.0 percent (weight/weight), and one or more agent selected from the group consisting of ethyl lauroyl arginate at a concentration of between about 0.02 and 0.05 percent (weight/weight), thymol at a concentration of between about 0.02 and 0.1 percent (weight/weight), galangal oil at a concentration of between about 0.02 and 0.05 percent (weight/weight), lemongrass oil at a concentration of between about 0.05 and 0.2 (weight/weight) and cinnamon oil (for example, cinnamon bark oil and/or cinnamon leaf oil) at a concentration of between about 0.0004 and 0.1 percent (weight/weight).

One non-limiting embodiment is a composition, for example a surface disinfectant composition for use in reducing the number and/or growth of microbes on an inanimate surface, comprising benzyl alcohol at a concentration of between about 0.5 and 1.0 percent (weight/weight), Biosecur® or other citrus extract at a concentration between about 0.5 and 1.0 percent (weight/weight), and one or more agent selected from the group consisting of ethyl lauroyl arginate at a concentration of between about 0.02 and 0.05 percent (weight/weight), lemongrass oil at a concentration of between about 0.02 and 0.2 (weight/weight), cedarwood oil at a concentration of between about 0.2 and 0.5 percent (weight/weight), pine oil at a concentration of between about 0.2 and 0.5 and cinnamon oil (for example, cinnamon bark oil and/or cinnamon leaf oil) at a concentration of between about 0.0004 and 0.1 percent (weight/weight).

One non-limiting embodiment is a composition, for example a preservative for addition to a personal care product, comprising benzyl alcohol at a concentration of between about 40 and 80 percent (weight/weight), citrus extract at a concentration of between about 40 and 80 percent (weight/weight), ethyl lauroyl arginate at a concentration of between about 2.5 and 10 percent (weight/weight), and one or more agent selected from the group consisting of thymol at a concentration of between about 3 and 5 percent (weight/weight), galangal oil at a concentration of between about 3 and 10 percent or between about 3 and 5 percent (weight/weight) and cinnamon oil (for example, cinnamon bark oil and/or cinnamon leaf oil) at a concentration of between about 4 and 10 percent (weight/weight).

One non-limiting embodiment is a composition, for example a personal care product, comprising benzyl alcohol at a concentration of between about 0.3-1.0 percent (weight/weight), citrus extract at a concentration between about 0.3-1.0 percent (weight/weight), ethyl lauroyl arginate at a concentration of between about 0.02-0.05 percent (weight/weight), and one or more agent selected from the group consisting of thymol oil at a concentration of between about 0.02-0.1 percent (weight/weight), galangal oil at a concentration of between about 0.02-0.1 percent or between about 0.02 and 0.15 percent (weight/weight) and cinnamon oil (for example, cinnamon bark oil and/or cinnamon leaf oil) at a concentration of between about 0.0004 and 0.1 percent or between about 0.1 and 0.2 percent (weight/weight).

One non-limiting embodiment is a composition, for example a preservative for addition to a food or beverage product, comprising benzyl alcohol at a concentration of between about 30 and 70 percent (weight/weight), citrus extract at a concentration of between about 20 and 30 percent (weight/weight), ethyl lauroyl arginate at a concentration of between about 1 and 2 percent (weight/weight), and one or more agent selected from the group consisting of thymol at a concentration of between about 4 and 10 percent (weight/weight), cinnamon oil (for example, cinnamon bark oil and/or cinnamon leaf oil) at a concentration of between about 4 and 10 percent (weight/weight) and galangal oil at a concentration of between about 4 and 10 percent (weight/weight).

One non-limiting embodiment is a composition, for example a food or beverage product, comprising benzyl alcohol at a concentration of between about 0.005 and 0.05 percent (weight/weight), citrus extract at a concentration between about 0.001 and 0.15 percent (weight/weight), ethyl lauroyl arginate at a concentration of between about 0.0001 and 0.02 percent (weight/weight) and one or more agent selected from the group consisting of thymol at a concentration of between about 0.0004 and 0.04 percent or between about 0.0004 and 0.1 percent (weight/weight), galangal oil at a concentration of between about 0.0004 and 0.04 percent or between about 0.02 and 0.15 percent (weight/weight) and cinnamon oil (for example, cinnamon bark oil and/or cinnamon leaf oil) at a concentration of between about 0.0004-0.04 percent or between about 0.004 and 0.1 percent (weight/weight).

One non-limiting embodiment is a composition, for example a food or beverage product, comprising benzyl alcohol at a concentration of between about 0.001 and 0.05 percent (weight/weight), citrus extract at a concentration between about 0.0001 and 0.15 percent (weight/weight), ethyl lauroyl arginate at a concentration of between about 0.001 and 0.2 percent (weight/weight), and one or more agent selected from the group consisting of thymol at a concentration of between about 0.0002 and 0.005 percent (weight/weight), galangal oil at a concentration of between about 0.0002 and 0.005 percent (weight/weight) and cinnamon oil (for example, cinnamon bark oil and/or cinnamon leaf oil) at a concentration of between about 0.0004-0.04 percent (weight/weight).

One non-limiting embodiment is a composition, for example a disinfectant composition for use in reducing the number and/or growth of microbes on a food product, comprising benzyl alcohol at a concentration of between about 30 and 80 percent (weight/weight), citrus extract at a concentration between about 10 and 50 percent (weight/weight), ethyl lauroyl arginate at a concentration of between about 0.2 and 2 percent or between about 2 and 20 percent (weight/weight), and one or more agent selected from the group consisting of thyme oil at a concentration of between about 2 and 20 percent (weight/weight), galangal oil at a concentration of between about 2 and 20 percent (weight/weight), rosemary oil at a concentration of between about 1 and 10 (weight/weight), cinnamon oil (for example, cinnamon bark oil and/or cinnamon leaf oil) at a concentration of between about 4 and 10 percent (weight/weight) and curry leaf oil at a concentration of between about 1 and 10 (weight/weight).

One non-limiting embodiment is a composition, for example a disinfectant composition for use in reducing the number and/or growth of microbes on a food product, comprising benzyl alcohol at a concentration of between about 0.5 and 1.0 percent (weight/weight), citrus extract at a concentration between about 0.5 and 1.0 percent (weight/weight), ethyl lauroyl arginate at a concentration of between about 0.001 and 0.05 percent (weight/weight), and one or more agent selected from the group consisting of thyme oil at a concentration of between about 0.01 and 0.5 percent or between about 0.001 and 0.5 percent (weight/weight), galangal oil at a concentration of between about 0.01 and 0.5 percent or between about 0.001 and 0.5 percent (weight/weight), rosemary oil at a concentration of between about 0.005 and 0.5 (weight/weight), cinnamon oil (for example, cinnamon bark oil and/or cinnamon leaf oil) at a concentration of between about 0.0004-0.1 percent or between about 0.05 and 0.2 percent (weight/weight) and curry leaf oil at a concentration of between about 0.005 and 0.5 (weight/weight).

One non-limiting embodiment is a composition, for example a topical disinfectant composition for use in reducing the number and/or growth of microbes on the skin of a subject, comprising benzyl alcohol at a concentration of between about 0.5 and 1.0 percent (weight/weight), citrus extract at a concentration between about 0.5 and 1.0 percent (weight/weight), ethyl lauroyl arginate at a concentration of between about 0.02 and 0.05 percent (weight/weight), and one or more agent selected from the group consisting of thyme oil at a concentration of between about 0.02 and 0.1 percent (weight/weight), galangal oil at a concentration of between about 0.02 and 0.05 percent (weight/weight), lemongrass oil at a concentration of between about 0.05 and 0.2 (weight/weight) and cinnamon oil (for example, cinnamon bark oil and/or cinnamon leaf oil) at a concentration of between about 0.0004 and 0.1 percent (weight/weight).

One non-limiting embodiment is a composition, for example a surface disinfectant composition for use in reducing the number and/or growth of microbes on an inanimate surface, comprising benzyl alcohol at a concentration of between about 0.5 and 1.0 percent (weight/weight), citrus extract at a concentration between about 0.5 and 1.0 percent (weight/weight), ethyl lauroyl arginate at a concentration of between about 0.02 and 0.05 percent (weight/weight) and one or more agent selected from the group consisting of lemongrass oil at a concentration of between about 0.02 and 0.2 (weight/weight), cedarwood oil at a concentration of between about 0.2 and 0.5 percent (weight/weight), pine oil at a concentration of between about 0.2 and 0.5 percent (weight/weight) and cinnamon oil (for example, cinnamon bark oil and/or cinnamon leaf oil) at a concentration of between about 0.0004 and 0.1 percent (weight/weight).

One non-limiting embodiment is a composition, for example an oral care composition for use in reducing the number and/or growth of microbes in the mouth of a subject, comprising benzyl alcohol at a concentration of between about 0.5 and 1.0 percent (weight/weight), citrus extract at a concentration between about 0.1 and 2.0 percent or between about 0.3 and 2 percent (weight/weight), optionally ethyl lauroyl arginate at a concentration of between about 0.05 and 0.2 percent or between about 0.005 and 0.02 percent (weight/weight), and one or more agent selected from the group consisting of clove bud oil at a concentration of between about 0.05 and 1.0 (weight/weight), peppermint oil at a concentration of between about 0.01 and 0.5 percent or between about 0.01 and 0.5 percent (weight/weight), thymol at a concentration of between about 0.05 and 1 percent (weight/weight), cinnamon oil (for example, cinnamon bark oil and/or cinnamon leaf oil) at a concentration of between about 0.05 and 0.2 percent (weight/weight), lemongrass oil at a concentration of between about 0-0.1 percent (weight/weight), rosemary oil at a concentration of between about 0.05 and 0.2 percent or between about 0.1 and 0.2 percent (weight/weight) and Echinacea purpurea extract at a concentration of between about 0.1 and 1 percent (weight/weight).

One non-limiting embodiment is a composition, for example an oral care composition for use in reducing the number and/or growth of microbes in the mouth of a subject, comprising benzyl alcohol at a concentration of between about 0.1 and 0.5 percent (weight/weight), Biosecur® or other citrus extract at a concentration between about 0.1 and 2.0 percent or between about 0.3 and 2 percent (weight/weight), and one or more agent selected from the group consisting of ethyl lauroyl arginate at a concentration of between about 0.02 and 0.2 percent or between about 0.005 and 0.02 percent (weight/weight), clove bud oil at a concentration of between about 0.05 and 1.0 (weight/weight), peppermint oil at a concentration of between about 0.01 and 0.5 percent or between about 0.01 and 0.5 percent or between about 0.02 and 0.1 percent (weight/weight), thymol at a concentration of between about 0.05 and 1 percent or 0.03 and 0.1 percent (weight/weight), cinnamon oil (for example, cinnamon bark oil and/or cinnamon leaf oil) at a concentration of between about 0.05 and 0.2 percent (weight/weight), lemongrass oil at a concentration of between about 0-0.1 percent (weight/weight), rosemary oil at a concentration of between about 0.05 and 0.2 percent or between about 0.1 and 0.2 percent (weight/weight), menthol at a concentration of between about 0.01 and 0.1 percent (weight/weight), Echinacea purpurea extract at a concentration of between about 0.1 and 1 percent (weight/weight), and an organic acid selected from the group consisting of benzoic, lactic acid and combinations thereof at a concentration of between about 0 and 0.2 percent (weight/weight).

One non-limiting embodiment is a composition comprising benzyl alcohol at a concentration between about 40 and 80 percent (weight/weight), Biosecur® or other citrus extract at a concentration between about 40 and 80 percent (weight/weight), ethyl lauryl arginate and/or glyceryl monolaurate at a concentration between about 0.5 and 2.0 percent (weight/weight), thymol and/or thyme oil at a concentration between about 3 and 5 percent (weight/weight), and organic acid selected from the group consisting of benzoic acid, lactic acid and combinations thereof at a concentration between about 0 and 20 percent (weight/weight).

One non-limiting embodiment is a composition comprising benzyl alcohol at a concentration between about 0.3 and 1.0 percent (weight/weight), Biosecur® or other citrus extract at a concentration between about 0.3 and 1.0 percent (weight/weight), ethyl lauryl arginate and/or glyceryl monolaurate at a concentration between about 0.02 and 0.05 percent (weight/weight), thymol and/or thyme oil at a concentration between about 0.02 and 0.1 percent (weight/weight), and organic acid selected from the group consisting of benzoic acid, lactic acid and combinations thereof at a concentration between about 0 and 0.2 percent (weight/weight).

One non-limiting embodiment is a composition comprising benzyl alcohol at a concentration between about 5 and 30 percent (weight/weight), Biosecur® or other citrus extract at a concentration between about 2 and 20 percent (weight/weight), ethyl lauryl arginate and/or glyceryl monolaurate at a concentration between about 1 and 5 percent (weight/weight), thymol and/or thyme oil at a concentration between about 0.1 and 0.4 percent (weight/weight), cinnamon bark oil at a concentration between about 2 and 10 percent (weight/weight), solvent such as ethanol and/or glycerine at a concentration between about 5 and 70 percent (weight/weight), and organic acid selected from the group consisting of benzoic acid, lactic acid and combinations thereof at a concentration between about 0 and 20 percent (weight/weight).

One non-limiting embodiment is a composition comprising benzyl alcohol at a concentration between about 0.01 and 0.06 percent (weight/weight), Biosecur® or other citrus extract at a concentration between about 0.004 and 0.04 percent (weight/weight), ethyl lauryl arginate and/or glyceryl monolaurate at a concentration between about 0.002 and 0.01 percent (weight/weight), thymol and/or thyme oil at a concentration between about 0.0002 and 0.0008 percent (weight/weight), cinnamon bark oil at a concentration between about 0.004 and 0.02 percent (weight/weight), solvent such as ethanol and/or glycerine at a concentration between about 0.01 and 0.15 percent (weight/weight), and organic acid selected from the group consisting of benzoic acid, lactic acid and combinations thereof at a concentration between about 0 and 0.2 percent (weight/weight).

One non-limiting embodiment is a composition comprising benzyl alcohol at a concentration between about 0.5 and 1.0 percent (weight/weight), Biosecur® or other citrus extract at a concentration between about 0.5 and 1.0 percent (weight/weight), ethyl lauryl arginate and/or glyceryl monolaurate at a concentration between about 0.001 and 0.05 percent (weight/weight), thymol and/or thyme oil at a concentration between about 0.001 and 0.5 percent (weight/weight), and organic acid selected from the group consisting of benzoic acid, lactic acid and combinations thereof at a concentration between about 0 and 0.2 percent (weight/weight).

One non-limiting embodiment is a composition comprising benzyl alcohol at a concentration between about 0.1 and 0.5 percent (weight/weight), Biosecur® or other citrus extract at a concentration between about 0.1 and 2.0 percent (weight/weight), ethyl lauryl arginate and/or glyceryl monolaurate at a concentration between about 0.02 and 0.2 percent (weight/weight), thymol and/or thyme oil at a concentration between about 0.03 and 0.1 percent (weight/weight), one or more flavoring agent selected from the group consisting of peppermint oil at a concentration between about 0.02 and 0.1 percent (weight/weight) and menthol at a concentration between about 0.01 and 0.1 percent (weight/weight), and organic acid selected from the group consisting of benzoic acid, lactic acid and combinations thereof at a concentration between about 0 and 0.2 percent (weight/weight).

One non-limiting embodiment is a composition comprising benzyl alcohol at a concentration between about 0.1 and 0.5 percent (weight/weight), Biosecur® or other citrus extract at a concentration between about 0.1 and 2.0 percent (weight/weight), thymol and/or thyme oil at a concentration between about 0.03 and 0.1 percent (weight/weight), one or more flavoring agent selected from the group consisting of peppermint oil at a concentration between about 0.02 and 0.1 percent (weight/weight) and menthol at a concentration between about 0.01 and 0.1 percent (weight/weight), and organic acid selected from the group consisting of benzoic acid, lactic acid and combinations thereof at a concentration between about 0 and 0.2 percent (weight/weight).

One non-limiting embodiment is a composition comprising benzyl alcohol at a concentration between about 10 and 70 percent (weight/weight), Biosecur® or other citrus extract at a concentration between about 1 and 80 percent (weight/weight), thymol and/or thyme oil at a concentration between about 0.5 and 20 percent (weight/weight), and organic acid selected from the group consisting of benzoic acid, lactic acid and combinations thereof at a concentration between about 0 and 20 percent (weight/weight).

One non-limiting embodiment is a composition comprising benzyl alcohol at a concentration between about 0.5 and 30 percent (weight/weight), Biosecur® or other citrus extract at a concentration between about 5 and 40 percent (weight/weight), ethyl lauryl arginate and/or glyceryl monolaurate at a concentration between about 0.2 and 4 percent (weight/weight), and organic acid selected from the group consisting of benzoic acid, lactic acid and combinations thereof at a concentration between about 0 and 20 percent (weight/weight).

One non-limiting embodiment is a composition comprising benzyl alcohol at a concentration between about 0.1 and 1 percent (weight/weight), Biosecur® or other citrus extract at a concentration between about 0.1 and 1 percent (weight/weight), ethyl lauryl arginate and/or glyceryl monolaurate at a concentration between about 0.05 and 0.5 percent (weight/weight), and organic acid selected from the group consisting of benzoic acid, lactic acid and combinations thereof at a concentration between about 0 and 0.2 percent (weight/weight).

One non-limiting embodiment is a composition comprising Biosecur® or other citrus extract at a concentration between about 5 and 40 percent (weight/weight), ethyl lauryl arginate and/or glyceryl monolaurate at a concentration between about 0.2 and 4 percent (weight/weight), thymol and/or thyme oil at a concentration between about 0.1 and 5 percent (weight/weight), and organic acid selected from the group consisting of benzoic acid, lactic acid and combinations thereof at a concentration between about 0 and 20 percent (weight/weight).

One non-limiting embodiment is a composition comprising Biosecur® or other citrus extract at a concentration between about 0.1 and 1 percent (weight/weight), ethyl lauryl arginate and/or glyceryl monolaurate at a concentration between about 0.02 and 0.5 percent (weight/weight), thymol and/or thyme oil at a concentration between about 0.01 and 0.5 percent (weight/weight), and organic acid selected from the group consisting of benzoic acid, lactic acid and combinations thereof at a concentration between about 0 and 0.2 percent (weight/weight).

In certain embodiments, any of the forgoing compositions may further comprise an organic acid, for example, but not limited to, benzoic acid, lactic acid or combinations thereof, wherein the organic acid is present at a concentration of between about 0 and 60 percent (weight/weight), or between about 0 an 50 percent (weight/weight), or between about 0 and 20 percent (weight/weight), or between about 0 and 10 percent (weight/weight). In certain embodiments, the organic acid is present at a concentration of between about 0.001 and 0.5 percent (weight/weight), or between about 0.005 and 0.4 percent (weight/weight), or between about 0.01 and 0.3 percent (weight/weight), or between about 0.05 and 0.3 percent (weight/weight), or between about 0.1 and about 0.2 percent (weight/weight). In certain embodiments, the organic acid is present at a concentration of between about 0.004 and about 0.04 percent (weight/weight), or between about 0 and 0.2 percent (weight/weight).

Any of the forgoing compositions may be used in methods of reducing the number and/or growth (inhibiting the growth) of microbes.

Any of the forgoing compositions may be prepared such that the citrus extract (e.g., Biosecur®) is added to a mixture of other components.

### 4. DETAILED DESCRIPTION

For purposes of clarity of description and not by way of limitation, this detailed description is divided into the following subsections:

(i) preservatives for personal care products;

(ii) preservatives for food and beverage products;

(iii) disinfectants for food;

(iv) topical disinfectants;

(v) surface disinfectants; and

(vi) oral formulations.

In certain embodiments, compositions disclosed herein comprise benzyl alcohol, at a concentration (percent weight/weight, “% w/w”) between about 0.005 and about 5.0% w/w, or between about 0.01 and about 4.0% w/w, or between about 0.1 and about 3.0% w/w, or between about 0.2 and about 2% w/w, or between about 0.3 and about 1% w/w. In specific non-limiting embodiments the benzyl alcohol is present in the composition at a concentration of between about 0.1 and about 5% w/w, or between about 0.1 and about 3% w/w, or between about 0.01 and about 0.06% w/w, or between about 0.1 and about 0.5% w/w, or between about 0.3 and about 1.0% w/w, or between about 0.5 and about 1.0 w/w, or between about 1.0 and about 3% w/w; or between about 0.5 and 2% w/w. In specific non-limiting embodiments the benzyl alcohol is plant-derived. The compositions described below may optionally further comprise phenoxy ethanol (0.3-1.0% w/w) and/or phenyl ethanol (0.3-1.0% w/w).

In certain embodiments, compositions disclosed herein comprise stock solutions that can be diluted for use comprising benzyl alcohol at a concentration between about 1 and about 90% w/w, or between about 5 and about 80% w/w, or between about 10 and about 50% w/w, or between about 20 and about 40% w/w. In specific non-limiting embodiments the benzyl alcohol is present in the stock at a concentration of between about 40 and about 80% w/w, or between about 5 and about 30% w/w.

In certain embodiments, compositions disclosed herein comprise Biosecur® or other citrus extract at a concentration (percent weight/weight, “% w/w”) between about 0.001 and about 5.0% w/w, or between about 0.01 and about 4.0% w/w, or between about 0.1 and about 3.0% w/w, or between about 0.2 and about 2% w/w, or between about 0.3 and about 1% w/w. In specific non-limiting embodiments the Biosecur® or other citrus extract is present in the composition at a concentration of between about 0.004 and about 0.04% w/w, or between about 0.1 and about 2.0% w/w, or between about 0.3 and about 1.0% w/w, or between about 0.5 and about 1.0% w/w.

In certain embodiments, compositions disclosed herein comprise stock solutions that can be diluted for use comprising Biosecur® or other citrus extract at a concentration between about 1 and about 90% w/w, or between about 5 and about 80% w/w, or between about 10 and about 50% w/w, or between about 20 and about 40% w/w. In specific non-limiting embodiments the Biosecur® or other citrus extract is present in the stock at a concentration of between about 40 and about 80% w/w, or between about 2 and about 20% w/w.

In certain embodiments, compositions disclosed herein comprise ethyl lauroyl arginate at a concentration (percent weight/weight, “% w/w”) between about 0.001 and about 1.0% w/w, or between about 0.002 and about 1.0% w/w, or between about 0.01 and about 0.5% w/w, or between about 0.02 and about 0.5% w/w. In specific non-limiting embodiments the ethyl lauroyl arginate is present in the composition at a concentration of between about 0.001 and about 0.05% w/w, or between about 0.002 and about 0.01% w/w, or between about 0.02 and about 0.05% w/w, or between about 0.02 and about 0.2% w/w.

In certain embodiments, compositions disclosed herein comprise stock solutions that can be diluted for use comprising ethyl lauroyl arginate at a concentration between about 0.1 and about 10% w/w, or between about 0.5 and about 8% w/w, or between about 1 and about 5% w/w. In specific non-limiting embodiments the ethyl lauroyl arginate is present in the stock at a concentration of between about 1 and about 5% w/w, or between about 0.5 and about 2% w/w.

### 4.1 Preservatives for Personal Care Products

Disclosed herein are compositions that may be comprised in personal care products to impart antimicrobial activity and accordingly act as preservatives.

In certain embodiments the composition is a stock solution comprising the recited active agents and a suitable solvent. Non-limiting examples of suitable solvents include water, ethanol, glycerin, octoxyglyverin, and mixtures thereof.

As a non-limiting example, the stock preservative can be incorporated into the final product at a use level of between about 0.7 and 1.2% weight/weight (w/w) (such that the preservative constitutes between about 0.7 and 1.2% (w/w) of the final product), or at a use level of between about 1.0 and 2.0% w/w, at a use level of about 1.5% w/w. Throughout this document, “% w/w stock” refers to the percent amount, weight to weight, in a stock solution, and “% w/w final” refers to the percent amount, weight to weight, in the final product.

In one non-limiting embodiment, the stock preservative comprises the following components:

| Ingredient | % w/w stock |
|---|---|
| Benzyl alcohol | 40-80 |
| Biosecur ®* | 40-80 |
| Ethyl lauroyl arginate | 0.5-2 |
| Thymol | 3-5 |

*or other citrus extract

In certain embodiments, the stock solution of preservative also comprises cinnamon oil (for example, cinnamon bark oil and/or cinnamon leaf oil) at a concentration of between about 4 and 10 percent (weight/weight).

In one non-limiting embodiment, the stock preservative comprises the following components:

| Ingredient | % w/w stock |
|---|---|
| Benzyl alcohol | 31.84 |
| Biosecur ®* | 63.69 |
| Ethyl lauroyl arginate | 1.27 |
| Thymol | 3.2 |

*or other citrus extract

In one non-limiting embodiment, the final composition comprises the following components which together have preservative activity:

| Ingredient | % w/w final |
|---|---|
| Benzyl alcohol | 0.3-1.0 |
| Biosecur ®* | 0.3-1.0 |
| Ethyl lauroyl arginate | 0.02-0.05 |
| Thyme oil | 0.02-0.1 |

*or other citrus extract

In certain embodiments, the final composition comprises cinnamon oil (for example, cinnamon bark oil and/or cinnamon leaf oil) at a concentration of between about 0.0004 and 0.1 percent or between about 0.1 and 0.2 percent (weight/weight).

In one non-limiting embodiment, the final composition comprises the following components which together have preservative activity:

| Ingredient | % w/w final |
|---|---|
| Benzyl alcohol | 0.5 |
| Biosecur ®* | 1.0 |
| Ethyl lauroyl arginate | 0.02 |
| Thyme oil | 0.05 |

*or other citrus extract

In one non-limiting embodiment, the stock preservative comprises the following components:

| Ingredient | % w/w stock |
|---|---|
| Benzyl alcohol | 40-80 |
| Biosecur ®* | 40-80 |
| Ethyl lauroyl arginate | 0.5-2 |
| Thymol | 3-5 |
| Galangal oil | 3-5 |

*or other citrus extract

In certain embodiments, the stock solution of preservative also comprises cinnamon oil (for example, cinnamon bark oil and/or cinnamon leaf oil) at a concentration of between about 4 and 10 percent (weight/weight).

In one non-limiting embodiment, the final composition comprises the following components which together have preservative activity:

| Ingredient | % w/w final |
|---|---|
| Benzyl alcohol | 0.3-1.0 |
| Biosecur ®* | 0.3-1.0 |
| Ethyl lauroyl arginate | 0.02-0.05 |
| Thyme oil | 0.02-0.1 |
| Galangal oil | 0.02-0.15 |

*or other citrus extract

In certain embodiments, the final composition comprises cinnamon oil (for example, cinnamon bark oil and/or cinnamon leaf oil) at a concentration of between about 0.0004 and 0.1 percent or between about 0.1 and 0.2 percent (weight/weight).

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of preservative comprises the following components. As a non-limiting example, the stock solution of preservative can be incorporated into the final product at a use level of between about 0.2 and 1.0% of the stock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.01 |

*or other citrus extract

In one non-limiting embodiment, a stock solution of preservative comprises the following components. As a non-limiting example, the stock solution of preservative can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Alcohol | 0.5-1.0 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of preservative comprises the following components. As a non-limiting example, the stock solution of preservative can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.3 |

*or other citrus extract

In one non-limiting embodiment, a stock solution of preservative comprises the following components. As a non-limiting example, the stock solution of preservative can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Lemongrass oil | 0.0005-0.01 |
| Glycerin | 0.005-0.3 |

In one non-limiting embodiment, a stock solution of preservative comprises the following components. As a non-limiting example, the stock solution of preservative can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Glycerin | 0.5-15.0 |

*or other citrus extract

In certain embodiments, the stock solution of preservative also comprises galangal oil at a concentration of between about 0.02 and 0.05 percent (weight/weight).

In certain embodiments, the stock solution of preservative also comprises lemongrass oil at a concentration of between about 0.1 and 1.0 percent (weight/weight).

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Glycerin | 0.0005-0.01 |

*or other citrus extract

In one non-limiting embodiment, the final composition comprises the following components which together have preservative activity:

| Ingredient | % w/w final |
|---|---|
| Benzyl alcohol | 0.3-2.0 |
| Biosecur ®* | 0.3-1.0 |
| Thymol | 0.02-0.1 |
| Glyceryl monolaurate | 0.02-0.5 |
| Phenyl ethanol | 0.1-1.0 |

*or other citrus extract

In one non-limiting embodiment, the final composition comprises the following components which together have preservative activity:

| Ingredient | % w/w final |
|---|---|
| Benzyl alcohol | 0.3 |
| Biosecur ®* | 0.5 |
| Thymol | 0.05 |
| Glyceryl monolaurate | 0.02 |
| Phenyl ethanol | 0.2 |

*or other citrus extract

In one non-limiting embodiment, the final composition comprises the following components which together have preservative activity:

| Ingredient | % w/w final |
|---|---|
| Benzyl alcohol | 0.3-2.0 |
| Biosecur ®* | 0.3-1.0 |
| Thymol | 0.02-0.1 |
| Glyceryl monolaurate | 0.02-0.5 |
| Phenyl ethanol | 0.1-1.0 |
| Galan gal oil | 0.02-0.15 |

*or other citrus extract

In one non-limiting embodiment, the final composition comprises the following components which together have preservative activity:

| Ingredient | % w/w final |
|---|---|
| Benzyl alcohol | 0.3 |
| Biosecur ®* | 0.5 |
| Thymol | 0.051 |
| Glyceryl monolaurate | 0.02 |
| Phenyl ethanol | 0.2 |
| Galan gal oil | 0.02 |

*or other citrus extract

In one non-limiting embodiment, the final composition comprises the following components which together have preservative activity:

| Ingredient | % w/w final |
|---|---|
| Phenyl ethanol | 0.1-1.0 |
| Thymol | 0.02-0.1 |
| Biosecur ®* | 0.3-1.0 |
| Glyceryl monolaurate | 0.02-0.5 |

*or other citrus extract

In one non-limiting embodiment, the final composition comprises the following components which together have preservative activity:

| Ingredient | % w/w final |
|---|---|
| Phenyl ethanol | 0.5 |
| Thymol | 0.05 |
| Biosecur ®* | 0.5 |
| Glyceryl monolaurate | 0.02 |

*or other citrus extract

In one non-limiting embodiment, the final composition comprises the following components which together have preservative activity:

| Ingredient | % w/w final |
|---|---|
| Phenyl ethanol | 0.1-1.0 |
| Thymol | 0.02-0.1 |
| Biosecurt* | 0.3-1.0 |
| Glyceryl monolaurate | 0.02-0.5 |
| Ethanol | 0.1-20.0 |

*or other citrus extract

In one non-limiting embodiment, the final composition comprises the following components which together have preservative activity:

| Ingredient | % w/w final |
|---|---|
| Phenyl ethanol | 0.5 |
| Thymol | 0.05 |
| Biosecur ®* | 0.5 |
| Glyceryl monolaurate | 0.02 |
| Ethanol | 0.1 |

*or other citrus extract

In one non-limiting embodiment, the stock preservative comprises the following components:

| Ingredient | % w/w stock |
|---|---|
| Biosecur ®* | 15-70 |
| Benzyl alcohol | 12.5-50 |
| Thymol | 0.5-20 |

* or other citrus extract

In one non-limiting embodiment, the stock preservative comprises the following components:

| Ingredient | % w/w stock |
|---|---|
| Biosecur ®* | 64.52 |
| Benzyl alcohol | 32.26 |
| Thymol | 3.22 |

*or other citrus extract

In one non-limiting embodiment, the stock preservative comprises the following components:

| Ingredient | % w/w stock |
|---|---|
| Benzyl alcohol | 12.5-50 |
| Thymol | 0.5-20 |
| Lemon grass oil | 0.4-20 |
| Leucidal advanced Bamboo extract** | 15-70 |
| Biosecure* | 1.0-5.0 |
| Ethanol | 0-40 |

*or other citrus extract
**(Leueonostoc/Arundinaria gigantea Leaf/Sorbus aucuparia fruit ferment filtrate)

In one non-limiting embodiment, the stock preservative comprises the following components:

| Ingredient | % w/w stock |
|---|---|
| Benzyl alcohol | 30.31 |
| Thymol | 3.03 |
| Lemon grass oil | 5.06 |
| Leucidal advanced Bamboo extract* * | 30.30 |
| Biosecur ®* | 1.0 |
| Ethanol | 30.30 |

*or other citrus extract
**(Leuconostoc/Arundinaria gigantea Leaf/Sorbus aucuparia fruit ferment filtrate).

In certain embodiments, the final composition comprises galangal oil at a concentration of between about 0.0002 and 0.005 percent (weight/weight).

In certain embodiments, the final composition comprises lemongrass oil at a concentration of between about 0.0005 and 0.01 percent (weight/weight).

In one non-limiting embodiment, the preservative comprises the following components:

| Ingredients | % w/w |
|---|---|
| Benzyl alcohol | 0.3-2.0 |
| Biosecur ®* | 0.3-1.0 |
| Thymol | 0.02-0.1 |
| Glyceryl monolaurate | 0.00-0.5 |
| Phenyl ethanol | 0.0-1.0 |
| Benzoic acid | 0.0-1.0 |

*or other citrus extract

In one non-limiting embodiment, the preservative comprises the following components:

| Ingredients | % w/w |
|---|---|
| Benzyl alcohol | 0.3 |
| Biosecur | 0.5 |
| Thymol | 0.05 |
| Glyceryl monolaurate | 0.02 |
| Phenyl ethanol | 0.2 |
| Benzoic acid | 0.1 |

*or other citrus extract

In one non-limiting embodiment, the preservative comprises the following components:

| Ingredients | % w/w |
|---|---|
| Benzyl alcohol | 0.5 |
| Biosecur ®* | 0.5 |
| Thymol | 0.05 |

*or other citrus extract

In one non-limiting embodiment, the preservative comprises the following components:

| Ingredients | % w/w |
|---|---|
| Benzyl alcohol | 0.5 |
| Biosecur ®* | 0.5 |
| Thymol | 0.05 |
| Glyceryl monolaurate | 0.02 |

*or other citrus extract

In one non-limiting embodiment, the preservative comprises the following components:

| Ingredients | % w/w |
|---|---|
| Benzyl alcohol | 0.5 |
| Biosecur ®* | 1.0 |
| Thymol | 0.05 |

*or other citrus extract

In one non-limiting embodiment, the preservative comprises the following components:

| Ingredients | % w/w |
|---|---|
| Benzyl alcohol | 0.5 |
| Biosecur ®* | 1.0 |
| Thymol | 0.05 |
| Glyceryl monolaurate | 0.02 |

*or other citrus extract

In one non-limiting embodiment, the preservative comprises the following components:

| Ingredients | % w/w |
|---|---|
| Benzyl alcohol | 0.5 |
| Biosecur ®* | 0.5 |
| Thymol | 0.05 |
| Benzoic acid | 0.1 |

*or other citrus extract

In one non-limiting embodiment, the preservative comprises the following components:

| Ingredients | % w/w |
|---|---|
| Benzyl alcohol | 0.5 |
| Biosecur ®* | 0.5 |
| Thymol | 0.05 |
| Glyceryl monolaurate | 0.02 |
| Benzoic acid | 0.1 |

*or other citrus extract

In one non-limiting embodiment, the preservative comprises the following components:

| Ingredients | % w/w |
|---|---|
| Benzyl alcohol | 0.5 |
| Biosecur ®* | 0.5 |
| Thymol | 0.05 |
| Benzoic acid | 0.1 |
| Phenyl ethanol | 0.2 |

*or other citrus extract

In one non-limiting embodiment, the preservative comprises the following components:

| Ingredients | % w/w |
|---|---|
| Benzyl alcohol | 0.5 |
| Biosecur ®* | 0.5 |
| Thymol | 0.05 |
| Glyceryl monolaurate | 0.02 |
| Benzoic acid | 0.1 |
| Phenyl ethanol | 0.2 |

*or other citrus extract

In one non-limiting embodiment, the preservative comprises the following components:

| Ingredients | % w/w |
|---|---|
| Biosecur ®* | 0.3-1.0 |
| Thymol | 0.02-0.1 |
| Glyceryl monolaurate | 0-0.5 |
| Phenyl ethanol | 0.1-1.0 |
| Benzoic acid | 0-1.0 |
| Alcohol | 0-1.0 |

*or other citrus extract

In one non-limiting embodiment, the preservative comprises the following components:

| Ingredients | % w/w |
|---|---|
| Biosecur ®* | 0.5 |
| Thymol | 0.05 |
| Glyceryl monolaurate | 0.02 |
| Phenyl ethanol | 0.2 |
| Benzoic acid | 0.1 |
| Alcohol | 0.1 |

*or other citrus extract

In one non-limiting embodiment, the preservative comprises the following components:

| Ingredients | % w/w |
|---|---|
| Phenyl ethanol | 0.3 |
| Biosecur ®* | 0.5 |
| Benzoic acid | 0.1 |
| Thymol | 0.05 |
| Alcohol | 0-0.3 |

*or other citrus extract

In one non-limiting embodiment, the preservative comprises the following components:

| Ingredients | % w/w |
|---|---|
| Biosecur ®* | 0.5 |
| Thymol | 0.05 |
| Benzoic acid | 0.1 |
| Alcohol | 0.1 |

*or other citrus extract

In particular non-limiting embodiments a preservative may comprise between about 0.3 and 0.7, or between about 0.3 and 1.0, or about 0.5 percent (here and elsewhere, unless stated otherwise percent is w/w) benzyl alcohol and between about 0.1 and 0.5, or between about 0.3 and 1.0 percent w/w Biosecur® or other citrus extract optionally combined with one or more of the following: lauroyl arginate (e.g. ethyl lauroyl arginate) at a concentration of between about 0.02 and 0.06 or between about 0.02 0.05 or about 0.04 percent, cinnamon oil (for example, cinnamon bark oil an/or cinnamon leaf oil) at a concentration of between about 0.1 and 0.2 or about 0.15 percent w/w; galangal oil at a concentration of between about 0.1 and 0.2 or about 0.15 percent w/w; thymol at a concentration of between about 0.02 and 0.1 or about 0.1 percent w/w, or a combination of thymol at a concentration of between about 0.05 and 0.1 percent w/w and galangal oil at a concentration of between about 0.1 and 0.2 or about 0.15 percent w/w. This preservative may be diluted to constitute between about 1.5 and 1.7 percent of a personal care product.

In one specific non-limiting embodiment the personal care product may be a lotion comprising the following components:

| Ingredient | % w/w |
|---|---|
| Water | 78.0 |
| Propylene glycol | 2.0 |
| Mineral oil | 9.5 |
| Ceteareth 25 | 2.5 |
| Cetearyl Alcohol | 4.0 |
| Glyceryl stearate | 2.0 |
| Monoglyceryl citrate | 2.0 |
| pH | 5.0 |

In one specific non-limiting embodiment the personal care product may be a lotion comprising the following components:

| Ingredient | % w/w |
|---|---|
| Water | 78.0 |
| Propylene glycol | 2.0 |
| Mineral oil | 9.5 |
| Ceteareth 25 | 2.5 |
| Cetearyl Alcohol | 4.0 |
| Glyceryl stearate | 2.0 |
| Monoglyceryl citrate | 2.0 |
| pH | 5.0 |

Provided herein is a method for producing a preservative or antimicrobial effect in a personal care product comprising incorporating a preservative formulation as set forth herein into the product.

Personal care products comprising the above ingredients include, but are not limited to, skin creams, lotions and splashes, hair care products, makeup (for example but not limited to foundation, powder, lipstick and eye makeup such as shadow, liner and mascara), veterinary cream, veterinary splash, etc.

### 4.2 Preservatives for Food and Beverage Products

Disclosed herein are compositions that may be comprised in personal care products to impart antimicrobial activity and accordingly act as preservatives.

In certain embodiments the composition is a stock solution comprising the recited active agents and a suitable solvent. Non-limiting examples of suitable solvents include water, ethanol, glycerin and octoxyglycerin and mixtures thereof.

In one non-limiting embodiment, a stock solution of preservative comprises the following components. As a non-limiting example, the stock solution of preservative can be incorporated into the final product at a use level of between about 1 and 10% w/w, or between about 0.1 and 0.5% w/w, or between about 0.1 and 0.3% w/w, or about 0.2% w/w.

In one non-limiting embodiment, the stock preservative comprises the following components:

| Ingredient | % w/w stock |
|---|---|
| Biosecur ®* | 5-40 |
| Benzyl alcohol | 0.0-30 |
| Thymol | 0.1-5 |
| Cinnamon oil | 1-20 |
| Glyceryl laurate | 0.2-4.0 |
| Ethanol | 0-80.0 |
| Glycerine | 0-60 |

*or other citrus extract

In one non-limiting embodiment, a food or beverage comprises the following components in the indicated final concentrations which impart preservative activity:

| Ingredient | % w/w in final product |
|---|---|
| Biosecur ®* | 0.5 |
| Benzyl alcohol | 1.0 |
| Thymol | 0.02 |
| Cinnamon oil | 0.5 |
| Glyceryl laurate | 0.1 |
| Ethanol | 1.5 |
| Glycerine | 1.38 |

*or other citrus extract

In one non-limiting embodiment, the stock preservative comprises the following components:

| Ingredient | % w/w stock |
|---|---|
| Benzyl alcohol | 5-30 |
| Biosecur ®* | 2-20 |
| Glyceryl laurate | 1-5 |
| Thymol | 0.1-0.4 |
| Cinnamon Bark oil | 2-10 |
| Solvent | 5-70 |

*or other citrus extract

In certain embodiments, the stock solution of preservative also comprises cinnamon oil (for example, cinnamon bark oil and/or cinnamon leaf oil) at a concentration of between about 4 and 10 percent (weight/weight).

In one non-limiting embodiment, a food or beverage comprises the following components in the indicated final concentrations which impart preservative activity:

| Ingredient | % w/w in final product |
|---|---|
| Benzyl alcohol | 0.01-0.06 |
| Biosecur ®* | 0.004-0.04 |
| Glyceryl laurate | 0.002-0.01 |
| Thymol | 0.0002-0.0008 |
| Cinnamon Bark oil | 0.01-0.04 |
| Solvent | 0.01-0.15 |

*or other citrus extract

In certain embodiments, the food or beverage also comprises cinnamon oil (for example, cinnamon bark oil and/or cinnamon leaf oil) at a concentration of between about 0.0004 and 0.1 percent (weight/weight).

In one non-limiting embodiment, a stock solution of preservative comprises the following components. As a non-limiting example, the stock solution of preservative can be incorporated into the final product at a use level of between about 1 and 10% w/w, or between about 0.1 and 0.3% w/w, or about 0.2% w/w.

| Ingredients | % w/w stock |
|---|---|
| Benzyl alcohol | 5-30 |
| Biosecur ®* | 2-20 |
| Cinnamon bark oil | 2-10 |
| Thymol | 0.1-0.4 |
| Glyceryl laurate | 1-5 |
| Ethanol | 5-70 |
| Glycerine | 5-40 |

*or other citrus extract

In certain embodiments, the stock solution of preservative also comprises cinnamon oil (for example, cinnamon bark oil and/or cinnamon leaf oil) at a concentration of between about 4 and 10 percent (weight/weight).

In one non-limiting embodiment, a food or beverage comprises the following components in the indicated final concentrations which impart preservative activity:

| Ingredients | % w/w in final product |
|---|---|
| Benzyl alcohol | 0.04 |
| Biosecur ®* | 0.01 |
| Cinnamon bark oil | 0.01 |
| Thymol | 0.0004 |
| Glyceryl laurate | 0.004 |
| Ethanol | 0.08 |
| Glycerine | 0.055 |

*or other citrus extract

In certain embodiments, the food or beverage also comprises cinnamon oil (for example, cinnamon bark oil and/or cinnamon leaf oil) at a concentration of between about 0.0004 and 0.1 percent (weight/weight).

In one non-limiting embodiment, a stock solution of preservative comprises the following components. As a non-limiting example, the stock solution of preservative can be incorporated into the final product at a use level of between about 1 and 10% w/w, or between about 0.1 and 0.3% w/w, or about 0.2% w/w.

| Ingredients | % w/w stock |
|---|---|
| Benzyl alcohol | 20 |
| Biosecur | 5 |
| Cinnamon bark oil | 5 |
| Thymol | 0.2 |
| Glyceryl laurate | 2 |
| Ethanol | 40 |
| Glyceine | 27.8 |

*or other citrus extract

In certain embodiments, the stock solution of preservative also comprises cinnamon oil (for example, cinnamon bark oil and/or cinnamon leaf oil) at a concentration of between about 4 and 10 percent (weight/weight).

In one non-limiting embodiment, a food or beverage comprises the following components in the indicated final concentrations which impart preservative activity:

| Ingredients | % w/w in final product |
|---|---|
| Benzyl alcohol | 0.04 |
| Biosecur ®* | 0.01 |
| Cinnamon bark oil | 0.01 |
| Thymol | 0.0004 |
| Glyceryl laurate | 0.004 |
| Ethanol | 0.08 |

*or other citrus extract

In certain embodiments, the food or beverage also comprises cinnamon oil (for example, cinnamon bark oil and/or cinnamon leaf oil) at a concentration of between about 0.0004 and 0.1 percent (weight/weight).

In one non-limiting embodiment, a stock solution of food preservative comprises the following components. As a non-limiting example, the stock solution of preservative can be incorporated into the final product at a use level of between about 0.2 and 1.0% of the stock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Galangal oil | 0.02-0.05 |
| Alcohol | 0.5-1.0 |
| Leniongrass oil | 0.05-1.0 |
| Glycerin | 0.5-15.0 |

*or other citrus extract

In one non-limiting embodiment, a stock solution of preservative comprises the following components. As a non-limiting example, the stock solution of preservative can be incorporated into the final product at a use level of between about 1 and 10% w/w

| Ingredient | % w/w stock |
|---|---|
| Benzyl alcohol | 10-30 |
| Biosecur ®* | 5-15 |
| Thymol | 2-10 |
| Galangal oil | 2-10 |

*or other citrus extract

In certain embodiments, the stock solution of preservative also comprises cinnamon oil (for example, cinnamon bark oil and/or cinnamon leaf oil) at a concentration of between about 4 and 10 percent (weight/weight).

In one non-limiting embodiment, a food or beverage comprises the following components in the indicated final concentrations which impart preservative activity:

| Ingredient | % w/w final |
|---|---|
| Benzyl alcohol | 0.1-0.2 |
| Biosecur ®* | 0.05-0.1 |
| Thymol | 0.02-0.05 |
| Galangal oil | 0.02-0.05 |

* or other citrus extract

In certain embodiments, the food or beverage also comprises cinnamon oil (for example, cinnamon bark oil and/or cinnamon leaf oil) at a concentration of between about 0.0004 and 0.1 percent (weight/weight).

In one non-limiting embodiment, a stock solution of preservative comprises the following components. As a non-limiting example, the stock solution of preservative can be incorporated into the final product at a use level of between about 1 and 10% w/w.

| Ingredient | % w/w stock |
|---|---|
| Benzyl alcohol | 10-20 |
| Biosecur ®* | 5-10 |
| Ethyl lauryl arginate | 0.5-20** |
| Thymol | 1-5 |
| Galangal oil | 1-5 |

*or other citrus extract
**for example 5-20 percent of a 10% solution of ethyl lauroyl arginate

In certain embodiments, the stock solution of preservative also comprises cinnamon oil (for example, cinnamon bark oil and/or cinnamon leaf oil) at a concentration of between about 4 and 10 percent (weight/weight).

In one non-limiting embodiment, a food or beverage comprises the following components in the indicated final concentrations which impart preservative activity:

| Ingredient | % w/w final |
|---|---|
| Benzyl alcohol | 0.1-0.2 |
| Biosecur ®* | 0.05-0.1 |
| Ethyl lauroyl arginate | 0.005-0.02 |
| Thymol | 0.02-0.05 |
| Galangal oil | 0.02-0.05 |

*or other citrus extract

In certain embodiments, the food or beverage also comprises cinnamon oil (for example, cinnamon bark oil and/or cinnamon leaf oil) at a concentration of between about 0.0004 and 0.1 percent (weight/weight).

In one non-limiting embodiment, a stock solution of preservative comprises the following components. As a non-limiting example, the stock solution of preservative can be incorporated into the final product at a use level of between about 1 and 10% w/w

| Ingredient | % w/w stock |
|---|---|
| Benzyl alcohol | 30-70 |
| Biosecur ®* | 20-30 |
| Ethyl lauryl arginate** | 1-20 |
| Thymol | 4-10 |

*or other citrus extract
**for example 10-20 percent of a 10% solution of ethyl lauroyl arginate

In certain embodiments, the stock solution of preservative also comprises one or more agent selected from the group consisting of cinnamon oil (for example, cinnamon bark oil and/or cinnamon leaf oil) at a concentration of between about 4 and 10 percent (weight/weight) and galangal oil at a concentration of between about 4 and 10 percent (weight/weight).

In one non-limiting embodiment, a food or beverage comprises the following components in the indicated final concentrations which impart preservative activity:

| Ingredient | % w/w final |
|---|---|
| Benzyl alcohol | 0.005-0.05 |
| Biosecur ®* | 0.001-0.15 |
| Ethyl lauroyl arginate | 0.0001-0.02 |
| Thymol | 0.0004-0.1 |

*or other citrus extract

In certain embodiments, the food or beverage also comprises one or more agent selected from the group consisting of cinnamon oil (for example, cinnamon bark oil and/or cinnamon leaf oil) at a concentration of between about 0.0004 and 0.1 percent or between about 0.0004 and 0.04 percent (weight/weight) and galangal oil at a concentration of between about 0.0004 and 0.04 percent or between about 0.02 and 0.15 percent (weight/weight).

In another non-limiting embodiment, a stock solution of preservative comprises the following components. As a non-limiting example, the stock solution of preservative can be incorporated into the final product at a use level of between about 0.01 and 0.05% w/w.

| Ingredient | % w/w stock |
|---|---|
| Benzyl alcohol | 60 |
| Biosecur ® | 20 |
| Ethyl lauroyl arginate | 16 |
| Thymol | 4 |

In certain embodiments, the stock solution of preservative also comprises cinnamon oil (for example, cinnamon bark oil and/or cinnamon leaf oil) at a concentration of between about 4 and 10 percent (weight/weight), wherein the cinnamon oil is incorporated into the final product at a concentration of between about 0.0004 and 0.1 percent (weight/weight).

In one non-limiting embodiment, the final food or beverage composition comprises the following components which together have preservative activity:

| Ingredient | % w/w final |
|---|---|
| Benzyl alcohol | 0.006-0.03 |
| Biosecur ® | 0.002-0.01 |
| Ethyl lauroyl arginate | 0.000016-0.00008 |
| Thymol | 0.0004-0.002 |

In another non-limiting embodiment, stock solution of preservative comprises the following components. As a non-limiting example, the stock solution of preservative can be incorporated into the final product at a use level of between about 0.01 and 0.05% w/w.

| Ingredient | % w/w stock |
|---|---|
| Benzyl alcohol | 30-70 |
| Biosecur ®* | 20-30 |
| Ethyl lauroyl arginate | 1-2 |
| Cinnamon oil | 4-10 |

*or other citrus extract

In one non-limiting embodiment, the final food or beverage composition comprises the following components which together have preservative activity:

| Ingredient | % w/w final |
|---|---|
| Benzyl alcohol | 0.005-0.05 |
| Biosecur ®* | 0.001-0.15 |
| Ethyl lauroyl arginate | 0.0001-0.02 |
| Cinnamon oil | 0.0004-0.04 |

*or other citrus extract

In one non-limiting embodiment, a stock solution of preservative comprises the following components. As a non-limiting example, the stock solution of preservative can be incorporated into the final product at a use level of between about 0.1 and 0.2% of the stock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Galangal oil | 0.02-0.05 |
| Alcohol | 0.5-1.0 |
| Lemongrass oil | 0.05-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of preservative comprises the following components. As a non-limiting example, the stock solution of preservative can be incorporated into the final product at a use level of between about 0.1 and 0.2% of the stock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.01 |

*or other citrus extract

In one non-limiting embodiment, a stock solution of preservative comprises the following components. As a non-limiting example, the stock solution of preservative can be incorporated into the final product at a use level of between about 0.1 and 0.2% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Alcohol | 0.5-1.0 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of preservative comprises the following components. As a non-limiting example, the stock solution of preservative can be incorporated into the final product at a use level of between about 0.1 and 0.2% of the stock k.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.3 |

*or other citrus extract

In one non-limiting embodiment, a stock solution of preservative comprises the following components. As a non-limiting example, the stock solution of preservative can be incorporated into the final product at a use level of between about 0.1 and 0.2% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Lemongrass oil | 0.0005-0.01 |
| Glycerin | 0.005-0.3 |

*or other citrus extract

In one non-limiting embodiment, a stock solution of preservative comprises the following components. As a non-limiting example, the stock solution of preservative can be incorporated into the final product at a use level of between about 0.1 and 0.2% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Glycerin | 0.5-15.0 |

*or other citrus extract

In certain embodiments, the stock solution of preservative also comprises galangal oil at a concentration of between about 0.02 and 0.05 percent (weight/weight).

In certain embodiments, the stock solution of preservative also comprises lemongrass oil at a concentration of between about 0.1 and 1.0 percent (weight/weight).

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Glycerin | 0.0005-0.01 |

*or other citrus extract

In one non-limiting embodiment, a stock solution of preservative comprises the following components. As a non-limiting example, the stock solution of preservative can be incorporated into the final product at a use level of between about 0.1 and 0.5% of the stock.

| Ingredient | % w/w stock |
|---|---|
| Biosecur ®* | 5-40 |
| Thymol | 0.1-5 |
| Cinnamon oil | 1-20 |
| Glyceryl laurate | 0.2-4.0 |
| Ethanol | 0-80.0 |
| Glycerine | 0-60 |

*or other citrus extract

In one non-limiting embodiment, the final composition comprises the following components which together have preservative activity:

| Ingredient | % w/w final |
|---|---|
| Biosecur ®* | 0.5 |
| Thymol | 0.02 |
| Cinnamon oil | 0.5 |
| Glyceryl laurate | 0.1 |
| Ethanol | 1.5 |
| Glycerine | 2.38 |

*or other citrus extract

In one non-limiting embodiment, a stock solution of preservative comprises the following components.

| Ingredient | % w/w stock |
|---|---|
| Biosecur ®* | 0.3-1.0 |
| Ethyl lauroyl arginate/Glyceryl laurate | 0.2-0.5 |
| Thymol | 0.02-0.1 |
| Cinnamon oil | 0.1-1.0 |
| Alcohol | 0.5-1.0 |
| Glycerine | 0.5-15.0 |
| Benzoic acid | 0-1.0 |

*or other citrus extract

In certain embodiments, the final composition comprises galangal oil at a concentration of between about 0.0002 and 0.005 percent (weight/weight).

In certain embodiments, the final composition comprises lemongrass oil at a concentration of between about 0.0005 and 0.01 percent (weight/weight),

In another non-limiting embodiment, a food or beverage product comprises benzyl alcohol at a concentration of between about 0.001 and 0.05 percent (weight/weight), Biosecur® at a concentration between about 0.0001 and 0.15 percent (weight/weight), and one or more agent selected from the group consisting of ethyl lauroyl arginate at a concentration of between about 0.0001 and 0.02 percent (weight/weight), thymol at a concentration of between about 0.0002 and 0.005 percent (weight/weight), galangal oil at a concentration of between about 0.0002 and 0.005 percent (weight/weight) and cinnamon oil (for example, cinnamon bark oil and/or cinnamon leaf oil) at a concentration of between about 0.0004-0.04 percent (weight/weight).

Provided herein is a method for producing a preservative or antimicrobial effect (i.e., inhibiting the growth of microbes) in a food or beverage product comprising incorporating a preservative formulation as set forth herein into the product. Microbes, the growth of which are inhibited, include but are not limited to one or more of Aspergillus niger, Candida albicans, Staphylococcus aureus, Staphylococcus epidermidis, Escherichia coli, Pseudomonas aeruginosa, Enterococcus, Streptococcus species and Salmonella species (e.g. typhimurium, enteritidis) and Listeria monocytogenes.

Food and beverage products comprising the above ingredients include, but are not limited to, juices such as apple, tomato, orange, pineapple, cranberry and grapefruit juice (and mixtures thereof), fruit cocktails, soft drinks, sodas, punches, canned fruits and vegetables, related products such as fruit cocktail and apple sauce, salad dressings, condiments such as catsup, and baby foods.

### 4.3 Disinfectants for Food

Disclosed herein are compositions that may be comprised in disinfectant compositions that may be used to reduce the number and/or growth of microbes on food products such as but not limited to vegetables, fruit, meat, poultry and fish. The disinfectant may be used by one or more of the original food producer or a food processor or the consumer. The food item may be washed, dipped, or soaked (for example, for between about 30 seconds and 1 hour or between about 30 seconds and 5 minutes) in the disinfectant composition, optionally followed by rinsing with water prior to consumption.

In certain embodiments the composition is a stock solution comprising the recited active agents and a suitable solvent. Non-limiting examples of suitable solvents include water, ethanol, glycerin, octoxyglycerin, and mixtures thereof.

As a non-limiting example, the stock disinfectant can be incorporated into the final product at a use level of between about 0.01 and 10 percent w/w.

In one non-limiting embodiment, a stock solution of food disinfectant comprises the following components.

| Ingredient | % w/w stock |
|---|---|
| Benzyl alcohol | 30-80 |
| Biosecur ®* | 10-50 |
| Thymol | 2-20 |
| Galangal oil | 2-20 |
| Rosemary oil | 1-10 |
| Curry leaf oil | 1-10 |

*or other citrus extract

In certain embodiments, the stock solution of preservative also comprises cinnamon oil (for example, cinnamon bark oil and/or cinnamon leaf oil) at a concentration of between about 4 and 10 percent (weight/weight).

In one non-limiting embodiment, a solution of food disinfectant at final dilution comprises the following components.

| Ingredient | % w/w final |
|---|---|
| Benzyl alcohol | 0.05-1.0 |
| Biosecur ®* | 0.05-1.0 |
| Thymol | 0.002-0.1 |
| Galangal oil | 0.002-0.1 |
| Rosemary oil | 0.005-0.2 |
| Curry leaf oil | 0.001-0.5 |

*or other citrus extract

In certain embodiments, the food or beverage also comprises cinnamon oil (for example, cinnamon bark oil and/or cinnamon leaf oil) at a concentration of between about 0.0004 and 0.1 percent or between about 0.05 and 0.2 percent (weight/weight).

In another non-limiting embodiment, a stock solution of food disinfectant comprises the following components. As a non-limiting example, the stock disinfectant can be incorporated into the final product at a use level of between about 0.5 and 10% w/w.

| Ingredient | % w/w stock |
|---|---|
| Benzyl alcohol | 30-80 |
| Biosecur ®* | 10-50 |
| Ethyl lauroyl arginate | 0.2-2 |
| Thymol | 2-20 |
| Galangal oil | 2-20 |
| Rosemary oil | 1-10 |
| Curry leaf oil | 1-10 |

*or other citrus extract

In certain embodiments, the stock solution of preservative also comprises cinnamon oil (for example, cinnamon bark oil and/or cinnamon leaf oil) at a concentration of between about 4 and 10 percent (weight/weight).

In another non-limiting embodiment, a solution of food disinfectant at final dilution comprises the following components

| Ingredient | % w/w final |
|---|---|
| Benzyl alcohol | 0.05-1.0 |
| Biosecur ® | 0.05-1.0 |
| Ethyl lauroyl arginate | 0.001-0.05 |
| Thymol | 0.01-0.5 |
| Galangal oil | 0.01-0.5 |
| Rosemary oil | 0.005-0.5 |
| Curry leaf oil | 0.005-0.5 |

In certain embodiments, the food or beverage also comprises cinnamon oil (for example, cinnamon bark oil and/or cinnamon leaf oil) at a concentration of between about 0.0004 and 0.1 percent or between about 0.05 and 0.2 percent (weight/weight).

In another non-limiting embodiment, a solution of food disinfectant at final dilution comprises the following components

| Ingredient | % w/w final |
|---|---|
| Benzyl alcohol | 0.5-1.0 |
| Biosecur ® | 0.5-1.0 |
| Ethyl lauroyl arginate | 0.001-0.05 |
| Thymol | 0.001-0.5 |
| Galangal oil | 0.001-0.5 |
| Rosemary oil | 0.005-0.5 |
| Curry leaf oil | 0.005-0.5 |

In certain embodiments, the food or beverage also comprises cinnamon oil (for example, cinnamon bark oil and/or cinnamon leaf oil) at a concentration of between about 0.0004 and 0.1 percent or between about 0.05 and 0.2 percent (weight/weight).

In another non-limiting embodiment, a solution of food disinfectant at final dilution comprises the following components

| Ingredient | % w/w final |
|---|---|
| Benzyl alcohol | 0.5-1.0 |
| Biosecur ® | 0.5-1.0 |
| Ethyl lauroyl arginate | 0.001-0.05 |
| Thymol | 0.001-0.5 |

In certain embodiments, the food or beverage also comprises cinnamon oil (for example, cinnamon bark oil and/or cinnamon leaf oil) at a concentration of between about 0.0004 and 0.1 percent or between about 0.05 and 0.2 percent (weight/weight).

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Galangal oil | 0.02-0.05 |
| Alcohol | 0.5-1.0 |
| Lemongrass oil | 0.05-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock tock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.01 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Alcohol | 0.5-1.0 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Lemongrass oil | 0.0005-0.01 |
| Glycerin | 0.005-0.3 |

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Glycerin | 0.5-15.0 |

*or other citrus extract

In certain embodiments, the stock solution of disinfectant also comprises galangal oil at a concentration of between about 0.02 and 0.05 percent (weight/weight).

In certain embodiments, the stock solution of disinfectant also comprises lemongrass oil at a concentration of between about 0.1 and 1.0 percent (weight/weight).

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Glycerin | 0.0005-0.01 |

*or other citrus extract

In certain embodiments, the final composition comprises galangal oil at a concentration of between about 0.0002 and 0.005 percent (weight/weight).

In certain embodiments, the final composition comprises lemongrass oil at a concentration of between about 0.0005 and 0.01 percent (weight/weight).

Disclosed herein is a method of reducing the number and/or growth of microbes in or on a food product comprising exposing the food product to a disinfectant composition as set forth herein. Microbes, the numbers and/or growth of which are reduced, include but are not limited to one or more of Aspergillus niger, Candida albicans, Staphylococcus aureus, Staphylococcus epidermidis, Escherichia coli, Pseudomonas aeruginosa, Enterococcus, Streptococcus species, Salmonella species (e.g. typhimurium, enteritidis) and Listeria monocytogenes.

### 4.4 Topical Disinfectants

Disclosed herein are compositions that may be comprised in disinfectant compositions that may be used to topically to reduce the number and/or growth of microbes on the skin of a human or non-human animal. The disinfectant may be a wash solution, a lotion, a cream, or a splash and/or may be comprised in a wipe.

The disinfectant composition comprises the recited active agents and a suitable solvent. Non-limiting examples of suitable solvents include water, ethanol, glycerin, octoxyglycerin, and mixtures thereof.

In one non-limiting embodiment, a topical disinfectant comprises the following components:

| Ingredient | % w/w |
|---|---|
| Benzyl alcohol | 0.5-1.0 |
| Biosecur ®* | 0.5-1.0 |
| Thyme oil | 0.02-0.1 |
| Galangal oil | 0.02-0.05 |
| Lemongrass oil | 0.05-0.2 |

*or other citrus extract

In certain embodiments, the topical disinfectant also comprises cinnamon oil (for example, cinnamon bark oil and/or cinnamon leaf oil) at a concentration of between about 0.0004 and 0.1 percent (weight/weight).

In another non-limiting embodiment, a topical disinfectant comprises the following components:

| Ingredient | % w/w |
|---|---|
| Benzyl alcohol | 0.5-1.0 |
| Biosecur ®* | 0.5-1.0 |
| Ethyl Lauroyl arginate | 0.02-0.05 |
| Thyme oil | 0.02-0.1 |
| Galangal oil | 0.02-0.05 |
| Lemongrass oil | 0.05-0.2 |

*or other citrus extract

In certain embodiments, the topical disinfectant also comprises cinnamon oil (for example, cinnamon bark oil and/or cinnamon leaf oil) at a concentration of between about 0.0004 and 0.1 percent (weight/weight).

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Galangal oil | 0.02-0.05 |
| Alcohol | 0.5-1.0 |
| Lemongrass oil | 0.05-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock tock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.01 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Alcohol | 0.5-1.0 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Lemongrass oil | 0.0005-0.01 |
| Glycerin | 0.005-0.3 |

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Glycerin | 0.5-15.0 |

*or other citrus extract

In certain embodiments, the stock solution of disinfectant also comprises galangal oil at a concentration of between about 0.02 and 0.05 percent (weight/weight).

In certain embodiments, the stock solution of disinfectant also comprises lemongrass oil at a concentration of between about 0.1 and 1.0 percent (weight/weight).

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Glycerin | 0.0005-0.01 |

*or other citrus extract

In certain embodiments, the final composition comprises galangal oil at a concentration of between about 0.0002 and 0.005 percent (weight/weight).

In certain embodiments, the final composition comprises lemongrass oil at a concentration of between about 0.0005 and 0.01 percent (weight/weight).

Disclosed herein is a method of reducing the number and/or growth of microbes on the skin of a subject comprising applying to the skin a topical disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia coli, Pseudomonas aeruginosa, Candida albicans and Listeria monocytogenes.

### 4.5 Surface Disinfectants

Disclosed herein are compositions that may be comprised in disinfectant compositions that may be used to topically to reduce the number and/or growth of microbes on inanimate surfaces, for use in disinfecting, for example and not by way of limitation, hard surfaces such as floors, countertops, sinks, toilets, shower stalls, tables, furniture, car interiors, baby furniture, etc. as well as soft surfaces but non-absorbent surfaces such as leather or vinyl.

The disinfectant composition comprises the recited active agents and a suitable solvent. Non-limiting examples of suitable solvents include water, ethanol, glycerin, octoxyglycerin, and mixtures thereof.

In one non-limiting embodiment, a surface disinfectant comprises the following components:

| Ingredient | % w/w |
|---|---|
| Benzyl alcohol | 0.5-1.0 |
| Biosecur ®* | 0.5-1.0 |
| Lemon grass oil | 0.02-2.0 |
| Cedarwood oil | 0.2-0.5 |
| Pine oil | 0.2-0.5 |
| Non ionic surfactant (e.g. Glucopon) | 3-6 |

*or other citrus extract

In certain embodiments, the surface disinfectant also comprises cinnamon oil (for example, cinnamon bark oil and/or cinnamon leaf oil) at a concentration of between about 0.0004 and 0.1 percent (weight/weight).

In another non-limiting embodiment, a surface disinfectant comprises the following components:

| Ingredient | % w/w |
|---|---|
| Benzyl alcohol | 0.5-1.0 |
| Biosecur ®* | 0.5-1.0 |
| Ethyl Lauroyl arginate | 0.02-0.05 |
| Lemon grass oil | 0.02-0.2 |
| Cedarwood oil | 0.2-0.5 |
| Pine oil | 0.2-0.5 |
| Non ionic surfactant (e.g. Glucopon) | 3-6 |

*or other citrus extract

In certain embodiments, the surface disinfectant also comprises cinnamon oil (for example, cinnamon bark oil and/or cinnamon leaf oil) at a concentration of between about 0.0004 and 0.1 percent (weight/weight).

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Galangal oil | 0.02-0.05 |
| Alcohol | 0.5-1.0 |
| Lemongrass oil | 0.05-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock tock.

| Ingredient | % w/w stock |
|---|---|
| Benzyl alcohol | 0.3-0.50 |
| Biosecur ®* | 0.3-1.0 |
| Ethyl lauroyl arginate/glyceryl laurate | 0.2-0.5 |
| Thymol | 0.02-0.1 |
| Cinnamon oil | 0.05-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Benzyl alcohol | 0.0015-0.0066 |
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.01 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Alcohol | 0.5-1.0 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Galangal oil | 0.0002-0.005 |
| Alcohol | 0.005-0.01 |
| Lemongrass oil | 0.0005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Alcohol | 0.5-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/glyceryl laurate | 0.001-0.01 |
| Thymol | 0.0002-0.02 |
| Cinnamon oil | 0.0005-0.01 |
| Alcohol | 0.005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/glyceryl laurate | 0.2-0.5 |
| Thymol | 0.02-0.1 |
| Cinnamon oil | 0.1-1.0 |
| Galangal oil | 0.02-0.05 |
| Lemongrass oil | 0.1-1.0 |
| Glycerin | 0.5-15.0 |

*or other citrus extract

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/ | 0.001-0.01 |
| glyceryl laurate | |
| Thymol | 0.0002-0.02 |
| Cinnamon oil | 0.0005-0.01 |
| Galangal oil | 0.0002-0.005 |
| Lemongrass oil | 0.0005-0.01 |
| Glycerin | 0.005-0.3 |

*or other citrus extract

In one non-limiting embodiment, a stock solution of disinfectant comprises the following components. As a non-limiting example, the stock solution of disinfectant can be incorporated into the final product at a use level of between about 0.2 and 1.0% of the stock.

| Ingredient | % w/w stock |
|---|---|
| Biosecur ®* | 0.3-1.0 |
| Ethyl lauroyl arginate/ | 0.2-0.5 |
| glyceryl laurate | |
| Thymol | 0.02-0.1 |
| Cinnamon oil | 0.1-1.0 |
| Glycerin | 0.5-15.0 |

*or other citrus extract

In certain embodiments, the stock solution of disinfectant also comprises galangal oil at a concentration of between about 0.02 and 0.05 percent (weight/weight).

In certain embodiments, the stock solution of disinfectant also comprises lemongrass oil at a concentration of between about 0.1 and 1.0 percent (weight/weight).

In one non-limiting embodiment, the final composition comprises the following components which together have preservative activity:

| Ingredient | % w/w final |
|---|---|
| Biosecur ®* | 0.0015-0.0132 |
| Ethyl lauroyl arginate/ | 0.001-0.01 |
| gyceryl laurate | |
| Thymol | 0.0002-0.02 |
| Cinnamon oil | 0.0005-0.01 |
| Glycerin | 0.0005-0.01 |

*or other citrus extract

In certain embodiments, the final composition comprises galangal oil at a concentration of between about 0.0002 and 0.005 percent (weight/weight).

In certain embodiments, the final composition comprises lemongrass oil at a concentration of between about 0.0005 and 0.01 percent (weight/weight).

Disclosed herein is a method of reducing the number and/or growth of microbes on an inanimate surface comprising applying to the surface a disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia coli, Pseudomonas aeruginosa, Candida albicans and Listeria monocytogenes.

The contents of the examples, including specific formulations, are hereby incorporated by reference into this detailed description.

### 4.6 Oral Formulations

In certain embodiments, the composition is an oral care composition for use in reducing the number and/or growth of microbes in the mouth of a subject comprising benzyl alcohol at a concentration of between about 0.1 and 1.0 percent, or between about 0.1 and 0.5 percent, or between about 0.5 and 1.0 percent (weight/weight), Biosecur® or other citrus extract at a concentration between about 0.1 and 2.0 percent or between about 0.3 and 2 percent (weight/weight), and one or more agent selected from the group consisting of ethyl lauroyl arginate at a concentration of between about 0.005 and 0.02 percent, or between about 0.05 and 2 percent, or between about 0.02 and 0.2 percent (weight/weight), clove bud oil at a concentration of between about 0.05 and 1.0 (weight/weight), peppermint oil at a concentration of between about 0.01 and 0.5 percent, or between about 0.02 and 0.1 percent (weight/weight), thymol at a concentration of between about 0.03 and 0.1 percent, or between about 0.05 and 1 percent or between about 0.05 and 0.1 percent (weight/weight), cinnamon oil (for example, cinnamon bark oil and/or cinnamon leaf oil) at a concentration of between about 0.05 and 0.2 percent (weight/weight), lemongrass oil at a concentration of between about 0-0.1 percent (weight/weight), menthol at a concentration of between about 0.01 and 1.0 percent, or between about 0.01 and 0.5 percent, or between about 0.01 and 0.1 percent (weight/weight) and rosemary oil at a concentration of between about 0.05 and 0.2 percent or between about 0.1 and 0.2 percent (weight/weight). This composition can further comprise Echinacea purpura extract at a concentration of between about 0.01 and 1.0 percent or between about 0.1 and 1 percent (weight/weight).

Also provided is a method of reducing the number and/or growth of microbes in the mouth of a subject comprising applying the oral care composition to an inside surface of the mouth of the subject.

As a non-limiting example, a stock oral care composition can be incorporated into the final product at a use level of between about 0.7 and 1.2% weight/weight (w/w) (such that the preservative constitutes between about 0.7 and 1.2% (w/w) of the final product), or at a use level of between about 1.0 and 2.0% w/w, at a use level of about 1.5% w/w.

In a specific non-limiting embodiment, the stock oral care composition has the following ingredients:

| Ingredient | % w/w stock |
|---|---|
| Water | 60-90 |
| Sorbitol | 0.1-1.0 |
| Pluronic F 87 Prill | 0.1-1.0 |
| Lactic acid/Benzoic acid | 0.05-1.0 |
| Biosecu ®* | 0.1-2.0 |
| Benzyl alcohol | 0.1-0.5 |
| Thymol | 0.03-0.1 |
| Menthol | 0.01-0.1 |
| Peppermint oil | 0.02-0.1 |
| Ethanol (95%) | 1-20 |
| Glyceryl monolaurate | 0.02-0.2 |
| Plantasol | 0.02-2.0 |

*or other citrus extract

In a specific non-limiting embodiment, the oral care composition has the following ingredients:

| Ingredient | % w/w |
|---|---|
| Water | 87.228 |
| Sorbitol | 0.3 |
| Pluronic F 87 Prill | 0.2 |
| Lactic acid/Benzoic acid | 0.1 |
| Biosecur ®* | 0.5 |
| Benzyl alcohol | 0.5 |
| Thymol | 0.07 |
| Menthol | 0.04 |
| Peppermint oil | 0.06 |
| Ethanol (95%) | 10 |
| Glyceryl monolaurate | 0.002 |
| Plantasol | 1 |

*or other citrus extract

In a specific non-limiting embodiment, the stock oral care composition has the following ingredients:

| Ingredient | % w/w stock |
|---|---|
| Water | 60-90 |
| Sorbitol | 0.1-1.0 |
| Pluronic F 87 Prill | 0.1-1.0 |
| Lactic acid/Benzoic acid | 0.05-1.0 |
| Biosecur ®* | 0.1-2.0 |
| Benzyl alcohol | 0.1-0.5 |
| Thymol | 0.03-0.1 |
| Menthol | 0.01-0.1 |
| Peppermint oil | 0.02-0.1 |
| Ethanol (95%) | 1-20 |
| Plantasol | 0.02-2.0 |

*or other citrus extract

In a specific non-limiting embodiment, the oral care composition has the following ingredients:

| Ingredient | % w/w |
|---|---|
| Water | 87.23 |
| Sorbitol | 0.3 |
| Pluronic F 87 Prill | 0.2 |
| Lactic acid | 0.1 |
| Biosecur ®* | 0.5 |
| Benzyl alcohol | 0.5 |
| Thymol | 0.07 |
| Menthol | 0.04 |
| Peppermint oil | 0.06 |
| Ethanol (95%) | 10 |
| Plantasol | 1 |

*or other citrus extract

In a specific non-limiting embodiment, the oral care composition has the following ingredients:

| Ingredient | % w/w |
|---|---|
| Benzyl alcohol | 0.5-1.0 |
| Thymol | 0.05-1.0 |
| Clove bud oil | 0.05-1.0 |
| Menthol | 0.01-1.0 |
| Peppermint oil | 0.01-0.5 |
| Propylene glycol | 0.1-3.0 |
| Pentyelene glycol | 0.05-1.0 |
| Glycerin | 1.0-20.0 |
| Biosecur ®* | 0.1-2.0 |
| Wasabi extract | 0.1-05 |
| Ethyl Lauroyl arginate | 0.05-0.2 |
| Water | 1.0-80.0 |
| Zinc Salicylate | 0.1-1.0 |
| Citric acid/Benzoic acid | 0.01-1.0 |
| Pluronic F-87 pril | 0.01-1.0 |
| Xylatol | 0.01-1.0 |
| Splenda | 0.01-1.0 |
| Echinacea Purpurea Extract | 0.01-1.0 |

*or other citrus extract

In a specific non-limiting embodiment, the oral care composition comprises the following ingredients:

| Ingredient | % w/w |
|---|---|
| Benzyl alcohol | 0.5-1.0 |
| Biosecur ®* | 0.3-2.0 |
| Ethyl lauroyl arginate | 0.05-0.2 |
| Thymol | 0.05-0.1 |
| Clove bud oil | 0.05-1.0 |
| Peppermint oil | 0.01-0.5 |
| Cinnamon oil | 0.05-0.2 |
| Lemongrass oil | 0.0-0.1 |
| Rosemary oil | 0.1-0.2 |
| Echinacea purpurea extract | 0.1-1.0 |

*or other citrus extract

Specific formulations described in the following example sections are hereby incorporated by reference into the detailed description.

### 5. EXAMPLE 1

A variety of preservative compositions were prepared and incorporated in a base anionic lotion and tested for their efficacy against Aspergillus niger. The base lotion had the following composition:

| Ingredient | % w/w |
|---|---|
| Water | 78.0 |
| Propylene glycol | 2.0 |
| Mineral oil | 9.5 |
| Ceteareth 25 | 2.5 |
| Cetearyl Alcohol | 4.0 |
| Glyceryl stearate | 2.0 |
| Monoglyceryl citrate | 2.0 |
| pH | 5.0 |

### Method of Preservative Efficacy Testing:

The testing method used in this study is based on the guidelines issued by the Cosmetics, Toiletries, and Fragrance Association, Inc. [CTFA, 2001] Briefly 1.5-1.7% of preservative (about 0.375 g) was added to 25 gms of the emulsion. 0.9 gm aliquots were dispensed into several culture tubes. They were divided into various groups and inoculated with 0.1 ml of A niger (10⁷ cfu/ml). After mixing, the tubes were incubated for 48 hours for bacteria and C. albicans and 7 days for A. niger respectively. The cultures were then diluted serially with Drug neutralizing fluid (DNF) and 0.5 ml aliquot plated on TSA and incubated for 24-48 hours (bacteria and C. albicans were incubated at 37° C. and A. niger at 30° C.). In the control group, the emulsion base without the preservatives was inoculated with each organism and processed similarly. The log 10 reduction in colony counts of the test group from the colony counts of control group was determined.

TABLE 1

| | | A. niger |
|---|---|---|
| | Composition | Log reduction from control growth |
| 1. | Benzyl alcohol (0.5) | 0.3 |
| 2. | Biosecur ® (2%) | 0.3 |
| 3. | Benzyl alcohol (0.5) | 1.26 |
| | Biosecur ® (0.3) | |
| 4. | Benzyl alcohol (0.5) | 2.25 |
| | Biosecur ® (0.3) | |
| | Cinnamon Leaf oil (0.15) | |
| 5. | Benzyl alcohol (0.5) | 2.14 |
| | Biosecur ® (0.3) | |
| | Galan gal oil (0.15) | |
| 6. | Benzyl alcohol (0.5) | 2.5 |
| | Biosecur ® (0.3) | |
| | Curry leaf oil (0.15) | |
| 7. | Benzyl alcohol (0.5) | 3.5 |
| | Biosecur ® (0.3) | |
| | Thymol (0.1) | |
| 8. | Benzyl alcohol (0.5) | 6.5 |
| | Biosecur ® (0.3) | |
| | Thymol (0.05) | |
| | Galan gal oil (0.15) | |
| 9. | Benzyl alcohol (0.5) | 6.5 |
| | Biosecur ®(0.3) | |
| | Cinnamon bark oil (0.15) | |
| | Control growth = 5 × 10⁶ − 7 × 10⁶ | |
| | (6.5 log10) | |

Conclusion:

Benzyl alcohol and Biosecur™ exhibits synergistic activity. All the essential oils tested enhanced the activity of Benzyl alcohol and Biosecur™. Cinnamon bark oil, thymol and a combination of thymol and galangal oil showed significant enhancement. However, the composition had an odor of essential oils.

### 6. EXAMPLE 2

Experiments were performed to test the antimicrobial effect of combinations of benzyl alcohol, Biosecur® and lauroyl arginate, in the context of a topical anionic lotion as described in Example 1. Testing was performed as in Example 1.

Although formulations comprising 1) benzyl alcohol, Biosecur™ and cinnamon bark oil or 2) benzyl alcohol, Biosecur®, thyme oil and galangal oil showed good antimicrobial efficacy against A. niger, they exhibited a distinct odor when incorporated into lotion. In order to mask this odor, additional agents were added. Surprisingly, ethyl lauroyl arginate (“Lauroyl arginate”) was found to enhance the antimicrobial activity (see Table 2).

TABLE 2

| | | A. niger |
|---|---|---|
| | Composition | Log reductionfrom control growth |
| 1. | Benzyl alcohol (0.5) | 0.3 |
| 2. | Biosecur ® (2%) | 0.3 |
| 3 | Ethyl Lauroyl Arginate (0.04) | 0.3 |
| 4. | Benzyl alcohol (0.5) | 1.26 |
| | Biosecur ® (0.3) | |
| 5. | Benzyl alcohol (0.5) | 3.4 |
| | Biosecur ® (0.3) | |
| | Ethyl Lauroyl Arginate (0.04) | |
| 6 | Thymol (0.05) | 0.3 |
| 7. | Benzyl alcohol (0.5) | 3.6 |
| | Biosecur ® (0.3) | |
| | Thymol (0.05) | |
| 8 . | Benzyl alcohol (0.3) | 3.6 |
| | Biosecur ® (0.5) | |
| | Ethyl Lauroyl Arginate (0.04) | |
| | Thymol (0.05) | |
| 9 | Benzyl alcohol (0.3) | 3.6 |
| | Biosecur ® (0.5) | |
| | Ethyl Lauroyl Arginate (0.04) | |
| | Galangal oil (0.15) | |
| 10 | Benzyl alcohol (0.5) | 6.5 |
| | Biosecur ® (0.3) | |
| | Ethyl Lauroyl Arginate (0.04) | |
| | Thymol (0.05) | |
| | Galangal oil (0.05) | |
| | Control growth 7.0 log10 | |

Conclusion:

In an ionic emulsion containing A. niger, benzyl alcohol and Biosecur® exhibits synergistic activity. Lauroyl arginate further enhances the preservative (antimicrobial) activity of benzyl alcohol and Biosecur® compositions. Similarly, thymol and galangal oil also enhance the preservative activity of benzyl alcohol and Biosecur®. When benzyl alcohol, Bisecur® and lauroyl arginate were combined with thymol or galangal oil individually, the activity was not found to be detectably increased. However, when these oils were used in combination with benzyl alcohol, Bisecur™ and lauroyl arginate, and a lower concentration of galangal oil to reduce odor, significant enhancement of activity was noticed. In Table 2, Group 9 appears to be the best combination for providing preservative antimicrobial activity.

### 7. EXAMPLE 3

Experiments were performed to study the effects of combinations of benzyl alcohol, Biosecur®, lauroyl arginate and thymol and/or Galangal oil for beverages and food.

Method of testing: the following ingredients and blends in the following % were prepared in apple juice and 5.0 ml of juice containing these concentrations were dispensed in culture tubes and inoculated with 10⁵ cfu/ml methicillin-resistant Staphylococcus aureus (“MRSA”). As a control, juice containing only bacteria was processed similarly. After 1 hour, serial dilutions were made with drug neutralizing fluid (DNF) and subcultured on TSA plates. The plates were incubated for 24-48 hours and colony counts were determined.

Because the presence of these components could potentially impact the taste or smell of foods and beverages, lower concentrations of ingredients were tested either alone and in combination.

TABLE 3

| | | MRSA |
|---|---|---|
| | Composition | Log reduction from control growth |
| 1. | Biosecur ® (0.1) | 0.0 |
| 2. | Ethyl Lauroyl arginate (0.02) | 0.0 |
| 3. | Benzyl alcohol (0.2) | 0.0 |
| 4. | Benzyl alcohol (0.2) | 0.5 |
| | Biosecur ® (0.1) | |
| 5. | Benzyl alcohol (0.2) | 1.8 |
| | Ethyl Lauroyl arginate (0.02) | |
| 6. | Biosecur ® (0.1) | 1.8 |
| | Ethyl Lauroyl arginate (0.02) | |
| 7. | Benzyl alcohol (0.2) | 2.5 |
| | Ethyl Lauroyl arginate(0.02) | |
| | Biosecur ® (0.1) | |
| 8. | Benzyl alcohol (0.2) | 7.0 |
| | Biosecur ® (0.1) | |
| | Ethyl Lauroyl arginate (0.02) | |
| | thymol (0.05) | |
| | Control growth 7 log10 | |

Conclusion:

In beverages, lauroyl arginate showed enhanced activity when used in combination with benzyl alcohol and Biosecur®. Addition of thymol significantly enhanced the activity.

### 8. EXAMPLE 4

Further to Example 3 above, the activity of blends of various agents in Apple juice against MRSA (10⁵/ml) was tested (1 hour post incubation). 1 to 10 dilution of the blends were tested in the apple juice. The results are shown below in Table 4.

TABLE 4

| | | MRSA |
|---|---|---|
| | Composition | Log reduction from control growth |
| 1. | Benzyl alcohol (0.2) | 2.5 |
| | Biosecur ® (0A) | |
| | Ethyl Lauroyl arginate (0.02) | |
| 2 | Benzyl alcohol (0,2) | 7.0 |
| | Biosecur ® (0.1) | |
| | Thymol (0.05) | |
| 3 | Benzyl alcohol (0.2) | 7.0 |
| | Ethyl Lauroyl arginate (0.01) | |
| | Thymol (0.02) | |
| | Galangal oil (0.02) | |

Conclusion:

Even though Group 2 is effective with 0.05% thyme oil, it has an odor. However when lauroyl arginate is used with a lower concentration of essential oil it is equally effective without odor. Group 3 has lower concentrations of essential oils and hence less odor and is effective against MRSA in beverages.

### 9. EXAMPLE 5

In the experiments described in this section, growth of microbes in beverages were tested after 7 hours and 24 hours. The stock solution containing the components as indicated in Table 5 were prepared and diluted 1 to 10 and 1 to 50 for testing. Table 5 shows the activity of blends of various agents in apple juice against Pseudomonas aeruginosa (10⁵/ml).

TABLE 5

| | | Pseudomonas aeruginosa | |
|---|---|---|---|
| | | Log reduction from control growth | |
| | Composition (% w/w) | 7 hrs | 24 hrs |
| 1. | Benzyl alcohol (0.01) | 2.45 | 4.5 |
| | Biosecur ® (0.005) | | |
| | Ethyl Lauroyl arginate (0.0005) | | |
| | Thymol (0.002) | | |
| 2. | Benzyl alcohol (0.002) | 2.45 | 4.5 |
| | Biosecur ® (0.001) | | |
| | Ethyl Lauroyl arginate (0.0001) | | |
| | Thymol (0.0004) | | |

Conclusion:

Low concentration of the blend containing Biosecur®, benzyl alcohol, lauroyl arginate and thymol prevented bacterial growth in apple juice beverage.

### 10. EXAMPLE 6

The following preservative composition was prepared as a stock solution and then diluted to constitute 0.025% w/w of apple juice:

| Ingredient | % w/w stock | % w/w final |
|---|---|---|
| Benzyl alcohol | 60 | 0.015 |
| Biosecur ® | 20 | 0.005 |
| Ethyl lauroyl arginate | 16 | 0.00004 |
| Thymol | 4 | .001 |

5.0 ml of the juice containing the preservative was distributed in culture tubes and inoculated with 10⁴ cfu of various microbes and incubated for 1-7 days. Bacterial growth was indicated by turbidity (“+”) whereas in the absence of bacterial growth the juice was clear (“−”). The results are shown in Table 6. For the control, plain juice without preservative was inoculated with the same culture.

TABLE 6

| | +/− Growth | |
|---|---|---|
| Microbe | Day 1 | Day 7 |
| A. niger | + | − |
| C .albicans | + | − |
| E. coli | + | − |
| P. aeruginosa | + | − |
| S .aureus | + | − |

Growth + turbid − Clear

All the culture tube i.e. control as well as preservative composition juice was inoculated with 10⁴ cfu bacteria and kept for incubation for 7 days and the results were recorded every 24 hrs.

### 11. EXAMPLE 7

The following preservative compositions were prepared.

| Ingredient | C8 (% w/w) | C13(% w/w) | B6(% w/w) |
|---|---|---|---|
| Benzyl alcohol | 57.5 | 66.0 | — |
| Ethyl Lauroyl arginate | 12.5 | 10 | 16.46 |
| Biosecur ® | 25.0 | 20.0 | 83.54 |
| Thymol | 5.0 | 4.0 | — |

Method: Apple juice containing 0.02% of C8, C13 or B6 preservative was prepared 5.0 ml of the juice was distributed in culture tubes and inoculated with 10² cfu of various microbes and incubated for 1-7 days. Microbial growth in the juice was noted. For control plain juice without preservative is inoculated with the same culture. The results are shown in Table 7.

TABLE 7

| | +/−Growth | | | | | | | |
|---|---|---|---|---|---|---|---|---|
| | Control | | C8 | | C13 | | B6 | |
| Microbe | D1 | D7 | D1 | D7 | D1 | D7 | D1 | D7 |
| A. niger | + | + | − | − | − | − | − | + |
| C. albicans | + | + | − | − | − | − | − | − |
| E. coli | + | + | − | − | − | − | − | − |
| P. aeruginosa | + | + | − | − | − | − | − | − |
| S. aureus | + | + | − | − | − | − | − | − |

+ Turbid (Growth)
− Clear (No growth)

### 12. EXAMPLE 8

The following preservative compositions were prepared.

| | % in stock solution | | |
|---|---|---|---|
| Ingredient | I | II | III |
| Benzyl alcohol | 33.334 | 0 | 0 |
| Biosecure ® | 2.0 | 4.04 | 4.209 |
| Cinnamon bark oil | 3.34 | 6.720 | 7.0 |
| Thymol | 0.133 | 0.268 | 0.280 |
| Galangal oil | 0.133 | 0.268 | 0.280 |
| 95% Ethanol | 57.06 | 80.64 | 84.03 |
| Lemongrass oil | 2.0 | 4.032 | 0 |
| Glyceryl laurate | 2.0 | 4.032 | 4.201 |

Method: Formulations 1, II and III above were added to apple juice at a concentration of 0.2%. 5.0 mL of juice containing formulations I, II or III was dispensed into separate culture tubes and inoculated with 0.1 mL of 10³ cfu/mL A. niger. For use as a control, juice containing the same concentration of A. niger was prepared. The tubes were observed daily for the growth of A. niger. The results of A. niger growth is shown in Table 8.

TABLE 8

Growth (Day)

| Formulation | 2 | 4 | 7 | 14 | 21 | 30 |
|---|---|---|---|---|---|---|
| Control | + | + | + | + | + | + |
| I | − | − | − | − | − | − |
| II | − | − | − | − | | |
| III | − | − | − | − | | |

+: Indicates growth of A. niger
−: Indicates no growth of A. niger

### Conclusion:

The antimicrobial effect of the compositions of the present application is maintained when the compositions are formulated with natural alcohol instead of benzyl alcohol.

### 13. EXAMPLE 9

A variety of preservative compositions were prepared and incorporated in a base lotion and tested for their efficacy against Aspergillus niger, Candida albicans, Staphylococcus aureus, and Pseudomonas aeruginosa. The base lotion had the following composition:

| Ingredient | % w/w |
|---|---|
| Water | 78.0 |
| Propylene glycol | 2.0 |
| Mineral oil | 9.5 |
| Ceteareth 25 | 2.5 |
| Cetearyl Alcohol | 4.0 |
| Glyceryl stearate | 2.0 |
| Monoglyceryl citrate | 2.0 |
| pH | 5.0 |

Method of preservative efficacy testing: The testing method used is based on the guidelines issued by the Cosmetics, Toiletries, and Fragrance Association, Inc. (CTFA, 2001). 1.5-1.7% of preservative was added to 25 g of the lotion. 0.9 g aliquots were dispensed into culture tubes. The culture tubes were inoculated with 0.1 ml of the test organism (S. aureus, P. aeruginosa and C. albicans (108 cfu/ml), and A. niger (107 cfu/ml)). After mixing, the tubes were incubated for 48 hours for S. aureus, P. aeruginosa and C. albicans, and 7 days for A. niger. The cultures were then diluted serially with Drug Neutralizing Fluid (DNF), and a 0.5 ml aliquot from each tube was plated on TSA and incubated for 24-48 hours (S. aureus, P. aeruginosa and C. albicans were incubated at 370° C., and A. niger at 300° C.). In the control group, the lotion base without the preservatives was inoculated with each organism and was processed similarly. The log 10 reduction in colony counts of the test group from the colony counts of the control group was determined.

The following preservative compositions were tested:

### Composition I

| Ingredient | % w/w stock | % w/w range |
|---|---|---|
| Biosecure ® | 64.53 | 15-70 |
| Benzyl alcohol | 32.25 | 12.5-50 |
| Thymol | 3.22 | 0.5-2.0 |

### Composition II

| Ingredient | % w/w stock | % w/w range |
|---|---|---|
| Benzyl alcohol | 30.31 | 12.5-50 |
| Thymol | 3.03 | 0.5-20 |
| Lemon grass oil | 5.06 | 0.4-20 |
| Leucidal advanced Bamboo extract* | 30.30 | 15-70 |
| Citrus extract | 1.0 | 1.0-5.0 |
| Ethanol | 30.30 | 0-40 |

*(Leuconostoc/Arundinaria gigantea Leaf/Sorbus aucuparia fruit ferment filtrate).

### Composition III

| Ingredient | % w/w stock | % w/w range |
|---|---|---|
| Benzyl alcohol | 26.31 | 12.5-50 |
| Thymol | 2.63 | 0.5-20 |
| Lemon grass oil | 2.63 | 0.5-20 |
| Octanediol | 26.35 | 10-50 |
| Wasabi extract | 15.78 | 15-70 |
| Citrus extract | 1.0 | 1.0-5.0 |
| Glycerin | 14.78 | 0-40 |
| Ethanol | 10.52 | 0-40 |

The results of the study are shown in Table 9.

TABLE 9
Log₁₀ reduction from control*

| | 24 hrs | | | 48 hrs | | | 7 days | | |
|---|---|---|---|---|---|---|---|---|---|
| Organism | 1 | 2 | 3 | 1 | 2 | 3 | 1 | 2 | 3 |
| S. aureus | 6.51 | 4.0 | 5.5 | ND | ND | ND | >7.0 | >7.0 | >7.0 |
| P. | 4.0 | 3.5 | 6.0 | ND | ND | ND | >7.0 | >7.0 | >7.0 |
| aeruginosa | | | | | | | | | |
| A. niger | ND | ND | ND | ND | ND | ND | 3.5 | 2.7 | 4.0 |
| C. albicans | ND | ND | ND | 6.60 | 4.0 | 6.5 | >7.0 | >7.0 | >7.0 |

### 14. EXAMPLE 10

A variety of preservative compositions were prepared and incorporated in a hydrophilic cream and tested for their efficacy against Aspergillus niger. The following preservative compositions were tested:

| | % w/w | |
|---|---|---|
| Ingredients | BS5 | BS5G |
| Benzyl alcohol | 0.5 | 0.5 |
| Biosecur | 0.5 | 0.5 |
| Thymol | 0.05 | 0.05 |
| Glyceryl | — | 0.02 |
| monolaurate | | |

| | % w/w | |
|---|---|---|
| Ingredients | BS6 | BS6G |
| Benzyl alcohol | 0.5 | 0.5 |
| Biosecur | 1.0 | 1.0 |
| Thymol | 0.05 | 0.05 |
| Glyceryl | — | 0.02 |
| monolaurate | | |

| | % w/w | |
|---|---|---|
| Ingredients | BS7 | BS7G |
| Benzyl alcohol | 0.5 | 0.5 |
| Biosecur | 0.5 | 0.5 |
| Thymol | 0.05 | 0.05 |
| Glyceryl | — | 0.02 |
| monolaurate | | |
| Benzoic acid | 0.1 | 0.1 |

| | % w/w | |
|---|---|---|
| Ingredients | BS8 | BS8G |
| Benzyl alcohol | 0.5 | 0.5 |
| Biosecur | 0.5 | 0.5 |
| Thymol | 0.05 | 0.05 |
| Glyceryl | — | 0.02 |
| monolaurate | | |
| Benzoic acid | 0.1 | 0.1 |
| Phenyl ethanol | 0.2 | 0.2 |

| | % w/w | |
|---|---|---|
| Ingredients | BS9 | BS9G |
| Benzyl alcohol | 0.5 | 0.5 |
| Biosecur | 0.5 | 0.5 |
| Thymol | 0.05 | 0.05 |
| Glyceryl | — | 0.02 |
| monolaurate | | |
| Benzoic acid | 0.1 | 0.1 |
| Phenyl ethanol | 0.2 | 0.2 |

Preparation of A. niger Culture: A. niger cultures grown on peptone dextrose agar slants were suspended in culture by scraping with a sterile loop. Peptone diluent was added to the slants and mixed to obtain a homogenous suspension that was transferred into a sterile culture tube and diluted with peptone to obtain 10⁷ cfu/ml.

Method of preservative efficacy testing: The testing method used in this study was based on the guidelines issued by the Cosmetics, Toiletries, and Fragrance Association, Inc. [CTFA, 2001] with slight modification. 1.5% of preservative was added to a hydrophilic cream and mixed well. 0.9 g aliquots were dispensed into several culture tubes. Aliquots were divided into various groups and inoculated with 0.1 ml of the test organism, A niger (10⁷ cfu/ml). After mixing, the tubes were incubated for 7 days. The cultures were then diluted serially with Drug Neutralizing Fluid (DNF), and 0.5 ml aliquots were plated on TSA and incubated for 24-48 hours (A. niger at 30° C.). In the control group, the cream base without the preservatives was inoculated with A. niger and processed similarly. The log₁₀ reduction in colony counts of the test group from the colony counts of control group was determined. For each group 3 samples were tested in one experiment. The experiment was repeated 3 times and the results are the mean of 9 samples. The results of the study are shown in Table 10.

TABLE 10
Log₁₀ reduction from control growth
(Control growth was 1 × 10⁷ − 5 × 10⁷)

| | Log₁₀ reduction from | |
|---|---|---|
| | control Growth | |
| Group | (7 day) | Use level (%) |
| BS5 | 3.5 | 1.5 |
| BS5G | 3.5 | 1.5 |
| BS6 | 6.5 | 1.5 |
| BS6G | 6.5 | 1.5 |
| BS7 | 6.5 | 1 |
| BS7G | 6.5 | 1 |
| BS8 | 6.5 | 1 |
| BS8G | 6.5 | 1 |
| BS9 | 6.5 | 1 |
| BS9G | 6.5 | 1 |

Conclusion: By adding benzoic acid to the preservative formulations, the antimicrobial efficacy was maintained, even when the concentration of Biosecur® was reduced. For example, formulations BS7 and BS7G included half the amount of Biosecur® as formulations BS6 and BS6G. However, BS7 and BS7G also included benzoic acid, and had the same antimicrobial efficacy as formulations BS6 and BS6G.

Various publications are cited herein, the contents of which are hereby incorporated by reference in their entireties.

What is claimed is:

1. An antimicrobial composition comprising
(i) benzyl alcohol;
(ii) citrus extract;
(iii) ethyl lauroyl arginate/glyceryl laurate; and
(iv) one or more agent selected from the group consisting of thymol; thyme oil; galangal oil; cinnamon oil; rosemary oil; curry leaf oil; lemongrass oil; cedarwood oil; pine oil; clove bud oil; peppermint oil; organic acid selected from the group consisting of benzoic acid, lactic acid and combinations thereof; and solvent.

2. The antimicrobial composition of claim 1, wherein the composition is formulated as a preservative for addition to a personal care product, comprising
(i) benzyl alcohol at a concentration of between about 40 and 80 percent (weight/weight);
(ii) citrus extract at a concentration of between about 40 and 80 percent (weight/weight);
(iii) ethyl lauroyl arginate/glyceryl laurate at a concentration of between about 0.5 and 2 percent (weight/weight); and
(iv) one or more agent selected from the group consisting of
(a) thymol at a concentration of between about 3 and 5 percent (weight/weight),
(b) galangal oil at a concentration of between about 3 and 10 percent (weight/weight),
(c) cinnamon oil at a concentration of between about 4 and 10 percent (weight/weight),
(d) organic acid selected from the group consisting of benzoic acid, lactic acid and combinations thereof at a concentration of between about 0 and 20 percent (weight/weight), and
(e) solvent selected from the group consisting of ethanol, glycerin, phenyl ethanol and combinations thereof.

3. The antimicrobial composition of claim 1, wherein the composition is formulated as a personal care product comprising
(i) benzyl alcohol at a concentration of between about 0.3 and 1.0 percent (weight/weight);
(ii) citrus extract at a concentration between about 0.3 and 1.0 percent (weight/weight);
(iii) ethyl lauroyl arginate/glyceryl laurate at a concentration of between about 0.02 and 0.05 percent (weight/weight); and
(iv) one or more agent selected from the group consisting of
(a) thymol at a concentration of between about 0.02 and 0.1 percent (weight/weight),
(b) galangal oil at a concentration of between about 0.02 and 0.15 percent (weight/weight),
(c) cinnamon oil at a concentration of between about 0.0004 and 0.1 percent (weight/weight), and
(d) organic acid selected from the group consisting of benzoic acid, lactic acid and combinations thereof at a concentration of between about 0 and 0.2 percent (weight/weight).

4. The antimicrobial composition of claim 1, wherein the composition is formulated as a preservative for addition to a food or beverage product comprising
(i) benzyl alcohol at a concentration of between about 30 and 70 percent (weight/weight);
(ii) citrus extract at a concentration of between about 20 and 30 percent (weight/weight);
(iii) ethyl lauroyl arginate/glyceryl laurate at a concentration of between about 1 and 2 percent (weight/weight); and
(iv) one or more agent selected from the group consisting of
(a) thymol at a concentration of between about 4 and 10 percent (weight/weight),
(b) galangal oil at a concentration of between about 4 and 10 percent (weight/weight),
(c) cinnamon oil at a concentration of between about 4 and 10 percent (weight/weight), and
(d) organic acid selected from the group consisting of benzoic acid, lactic acid and combinations thereof at a concentration of between about 0 and 20 percent (weight/weight).

5. The antimicrobial composition of claim 1, wherein the composition is formulated as a food or beverage product comprising
(i) benzyl alcohol at a concentration of between about 0.005 and 0.05 percent (weight/weight);
(ii) citrus extract at a concentration between about 0.001 and 0.15 percent (weight/weight);
(iii) ethyl lauroyl arginate/glyceryl laurate at a concentration of between about 0.0001 and 0.02 percent (weight/weight); and
(iv) one or more agent selected from the group consisting of
(a) thymol at a concentration of between about 0.0004 and 0.1 percent (weight/weight),
(b) galangal oil at a concentration of between about 0.0004 and 0.04 percent (weight/weight),
(c) cinnamon oil at a concentration of between about 0.0004 and 0.04 percent (weight/weight), and
(d) organic acid selected from the group consisting of benzoic acid, lactic acid and combinations thereof at a concentration of between about 0.004 and 0.04 percent (weight/weight).

6. The antimicrobial composition of claim 1, wherein the composition is formulated as a food or beverage product comprising
(i) benzyl alcohol at a concentration of between about 0.001 and 0.05 percent (weight/weight);
(ii) citrus extract at a concentration between about 0.0001 and 0.15 percent (weight/weight);
(iii) ethyl lauroyl arginate/glyceryl laurate at a concentration of between about 0.001 and 0.2 percent (weight/weight); and
(iv) one or more agent selected from the group consisting of
(a) thymol at a concentration of between about 0.0002 and 0.005 percent (weight/weight),
(b) galangal oil at a concentration of between about 0.0002 and 0.005 percent (weight/weight),
(c) cinnamon oil at a concentration of between about 0.0004 and 0.04 percent (weight/weight), and
(d) organic acid selected from the group consisting of benzoic acid, lactic acid and combinations thereof at a concentration of between about 0.004 and 0.04 percent (weight/weight).

7. The antimicrobial composition of claim 1, wherein the composition is formulated as a preservative for addition to a food or beverage product comprising
(i) benzyl alcohol at a concentration of between about 5 and 30 percent (weight/weight);
(ii) citrus extract at a concentration between about 2 and 20 percent (weight/weight);
(iii) ethyl lauroyl arginate/glyceryl laurate at a concentration of between about 1 and 5 percent (weight/weight); and
(iv) one or more agent selected from the group consisting of
(a) thymol at a concentration of between about 0.1 and 0.4 percent (weight/weight),
(b) cinnamon oil at a concentration of between about 2 and 10 percent (weight/weight),
(c) organic acid selected from the group consisting of benzoic acid, lactic acid and combinations thereof at a concentration of between about 0 and 20 percent (weight/weight), and
(d) solvent at a concentration of between about 5 and 70 percent (weight/weight).

8. The antimicrobial composition of claim 1, wherein the composition is formulated as a food or beverage product comprising
(i) benzyl alcohol at a concentration of between about 0.01 and 0.06 percent (weight/weight);
(ii) citrus extract at a concentration between about 0.004 and 0.04 percent (weight/weight);
(iii) ethyl lauroyl arginate/glyceryl laurate at a concentration of between about 0.002 and 0.01 percent (weight/weight); and
(iv) one or more agent selected from the group consisting of
(a) thymol at a concentration of between about 0.0002 and 0.0008 percent (weight/weight),
(b) cinnamon oil at a concentration of between about 0.004 and 0.02 percent (weight/weight),
(c) organic acid selected from the group consisting of benzoic acid, lactic acid and combinations thereof at a concentration of between about 0.004 and 0.04 percent (weight/weight), and
(d) solvent at a concentration of between about 0.01 and 0.15 percent (weight/weight).

9. The antimicrobial composition of claim 1, wherein the composition is formulated as a disinfectant composition for use in reducing the number and/or growth of microbes on a food product comprising
(i) benzyl alcohol at a concentration of between about 30 and 80 percent (weight/weight);
(ii) citrus extract at a concentration between about 10 and 50 percent (weight/weight);
(iii) ethyl lauroyl arginate/glyceryl laurate at a concentration of between about 0.2 and 2 percent (weight/weight); and
(iv) one or more agent selected from the group consisting of
(a) thyme oil at a concentration of between about 2 and 20 percent (weight/weight),
(b) galangal oil at a concentration of between about 2 and 20 percent (weight/weight),
(c) rosemary oil at a concentration of between about 1 and 10 (weight/weight),
(d) curry leaf oil at a concentration of between about 1 and 10 (weight/weight),
(e) cinnamon oil at a concentration of between about 4 and 10 percent (weight/weight), and
(f) organic acid selected from the group consisting of benzoic acid, lactic acid and combinations thereof at a concentration of between about 0 and 20 percent (weight/weight).

10. The antimicrobial composition of claim 1, wherein the composition is formulated as a disinfectant composition for use in reducing the number and/or growth of microbes on a food product comprising
(i) benzyl alcohol at a concentration of between about 0.5 and 1.0 percent (weight/weight);
(ii) citrus extract at a concentration between about 0.5 and 1.0 percent (weight/weight);
(iii) ethyl lauroyl arginate/glyceryl laurate at a concentration of between about 0.001 and 0.05 percent (weight/weight); and
(iv) one or more agent selected from the group consisting of
(a) thymol at a concentration of between about 0.001 and 0.5 percent (weight/weight),
(b) galangal oil at a concentration of between about 0.001 and 0.5 percent (weight/weight),
(c) rosemary oil at a concentration of between about 0.005 and 0.5 percent (weight/weight),
(d) curry leaf oil at a concentration of between about 0.005 and 0.5 (weight/weight),
(e) cinnamon oil at a concentration of between about 0.05 and 0.2 percent (weight/weight), and
(f) organic acid selected from the group consisting of benzoic acid, lactic acid and combinations thereof at a concentration of between about 0 and 0.2 percent (weight/weight).

11. The antimicrobial composition of claim 1, wherein the composition is formulated as a topical disinfectant composition for use in reducing the number and/or growth of microbes on the skin of a subject comprising
(i) benzyl alcohol at a concentration of between about 0.5 and 1.0 percent (weight/weight);
(ii) citrus extract at a concentration between about 0.5 and 1.0 percent (weight/weight);
(iii) ethyl lauroyl arginate/glyceryl laurate at a concentration of between about 0.02 and 0.05 percent (weight/weight); and
(iv) one or more agent selected from the group consisting of
(a) thyme oil at a concentration of between about 0.02 and 0.1 percent (weight/weight),
(b) galangal oil at a concentration of between about 0.02 and 0.05 percent (weight/weight),
(c) lemongrass oil at a concentration of between about 0.05 and 0.2 (weight/weight),
(d) cinnamon oil at a concentration of between about 0.0004 and 0.1 percent (weight/weight), and
(e) organic acid selected from the group consisting of benzoic acid, lactic acid and combinations thereof at a concentration of between about 0 and 0.2 percent (weight/weight).

12. The antimicrobial composition of claim 1, wherein the composition is formulated as a surface disinfectant composition for use in reducing the number and/or growth of microbes on an inanimate surface comprising
(i) benzyl alcohol at a concentration of between about 0.5 and 1.0 percent (weight/weight);
(ii) citrus extract at a concentration between about 0.5 and 1.0 percent (weight/weight);
(iii) ethyl lauroyl arginase/glyceryl laurate at a concentration of between about 0.02 and 0.05 percent (weight/weight); and
(iv) one or more agent selected from the group consisting of
(a) lemongrass oil at a concentration of between about 0.02 and 0.2 (weight/weight),
(b) cedarwood oil at a concentration of between about 0.2 and 0.5 percent (weight/weight),
(c) pine oil at a concentration of between about 0.2 and 0.5 percent (weight/weight),
(d) cinnamon oil at a concentration of between about 0.0004 and 0.1 percent (weight/weight), and
(e) organic acid selected from the group consisting of benzoic acid, lactic acid and combinations thereof at a concentration of between about 0 and 0.2 percent (weight/weight).

13. The antimicrobial composition of claim 12, which further comprises a nonionic surfactant.

14. The antimicrobial composition of claim 1, wherein the composition is formulated as an oral care composition for use in reducing the number and/or growth of microbes in the mouth of a subject comprising
(i) benzyl alcohol at a concentration of between about 0.5 and 1.0 percent (weight/weight);
(ii) citrus extract at a concentration between about 0.1 and 2.0 percent (weight/weight);
(iii) ethyl lauroyl arginate/glyceryl laurate at a concentration of between about 0.05 and 0.2 percent (weight/weight); and
(iv) one or more agent selected from the group consisting of
(a) clove bud oil at a concentration of between about 0.05 and 1.0 (weight/weight),
(b) peppermint oil at a concentration of between about 0.01 and 0.5 percent (weight/weight),
(c) thymol at a concentration of between about 0.05 and 1 percent (weight/weight),
(d) cinnamon oil at a concentration of between about 0.05 and 0.2 percent (weight/weight),
(e) lemongrass oil at a concentration of between about 0-0.1 percent (weight/weight), and
(f) rosemary oil at a concentration of between about 0.1 and 0.2 percent (weight/weight).

15. The antimicrobial composition of claim 14, which further comprises Echinacea purpura extract at a concentration of between about 0.01 and 1.0 percent (weight/weight).

16. An oral care composition for use in reducing the number and/or growth of microbes in the mouth of a subject comprising
(i) benzyl alcohol at a concentration of between about 0.1 and 0.5 percent (weight/weight);
(ii) citrus extract at a concentration between about 0.1 and 2.0 percent (weight/weight); and
(iii) one or more agent selected from the group consisting of
(a) ethyl lauroyl arginate/glyceryl laurate at a concentration of between about 0.02 and 0.2 percent (weight/weight),
(b) clove bud oil at a concentration of between about 0.05 and 1.0 (weight/weight),
(c) peppermint oil at a concentration of between about 0.02 and 0.1 percent (weight/weight),
(d) thymol at a concentration of between about 0.03 and 0.1 percent (weight/weight),
(e) cinnamon oil at a concentration of between about 0.05 and 0.2 percent (weight/weight),
(f) lemongrass oil at a concentration of between about 0 and 0.1 percent (weight/weight),
(g) menthol at a concentration of between about 0.01 and 0.1 percent (weight/weight), and
(h) rosemary oil at a concentration of between about 0.1 and 0.2 percent (weight/weight).

17. An antimicrobial composition comprising
(i) alcohol selected from the group consisting of benzyl alcohol, phenyl ethanol, ethanol and combinations thereof;
(ii) Biosecur® or other citrus extract;
(iii) ethyl lauroyl arginate/glyceryl laurate; and
(iv) one or more agent selected from the group consisting of thymol, thyme oil, galangal oil, cinnamon oil, lemongrass oil, orange oil, rosemary oil, curry leaf oil, cedarwood oil, pine oil, peppermint oil, menthol, aliphatic alcohol, phenoxy ethanol, phenyl ethanol, and organic acid selected from the group consisting of benzoic acid, lactic acid and combinations thereof.

18. The antimicrobial composition of claim 17, comprising
(i) alcohol selected from the group consisting of benzyl alcohol, phenyl ethanol, ethanol and combinations thereof at a concentration of between about 0.1 and 2.0 percent weight/weight (w/w);
(ii) Biosecur® or other citrus extract at a concentration of between about 0.3 and 1.0% w/w;
(iii) ethyl lauroyl arginate/glyceryl laurate at a concentration of between about 0.02 and 0.05% w/w; and
(iv) one or more agent selected from the group consisting of
(a) thymol at a concentration of between about 0.02 and 0.1 percent w/w,
(b) galangal oil at a concentration of between about 0.02 and 0.15 percent w/w,
(c) cinnamon oil at a concentration of between about 0.05 and 0.2 percent w/w,
(d) lemongrass oil at a concentration of between about 0.1 and 0.5 percent w/w,
(e) orange oil at a concentration of between about 0.1 and 0.2 percent w/w,
(f) rosemary oil at a concentration of between about 0.1 and 0.2 percent w/w,
(g) curry leaf oil at a concentration of between about 0.05 and 0.5 percent w/w,
(h) cedarwood oil at a concentration of between about 0.2 and 0.5 percent w/w,
(i) pine oil at a concentration of between about 0.1 and 0.5 percent w/w,
(j) aliphatic alcohol at a concentration of between about 5 and 20 percent w/w,
(k) phenoxy ethanol at a concentration of between about 0.3 and 1.0 percent w/w,
(l) phenyl ethanol at a concentration of between about 0.3 and 1.0 percent w/w, and
(m) organic acid selected from the group consisting of benzoic acid, lactic acid and combinations thereof.

19. The antimicrobial composition of claim 17, wherein the composition is formulated as a preservative for addition to a personal care product comprising
(i) alcohol selected from the group consisting of benzyl alcohol, ethanol, phenyl ethanol and combinations thereof at a concentration of between about 40 and 80 percent (weight/weight);
(ii) Biosecur® or other citrus extract at a concentration of between about 40 and 80 percent (weight/weight);
(iii) ethyl lauroyl arginate/glyceryl laurate at a concentration of between about 0.5 and 2 percent (weight/weight); and
(iv) one or more agent selected from the group consisting of
(a) thymol at a concentration of between about 3 and 5 percent (weight/weight),
(b) galangal oil at a concentration of between about 3 and 10 percent (weight/weight), and
(c) cinnamon oil at a concentration of between about 4 and 10 percent (weight/weight).

20. The antimicrobial composition of claim 17 comprising
(i) benzyl alcohol at a concentration between about 40 and 80 percent (weight/weight);
(ii) Biosecur® or other citrus extract at a concentration between about 40 and 80 percent (weight/weight);
(iii) ethyl lauroyl arginate/glyceryl laurate at a concentration between about 0.5 and 2.0 percent (weight/weight);
(iv) an agent selected from the group consisting of thymol, thyme oil, and combinations thereof, at a concentration between about 3 and 5 percent (weight/weight); and
(v) organic acid selected from the group consisting of benzoic acid, lactic acid and combinations thereof, at a concentration between about 0 and 20 percent (weight/weight).

21. The antimicrobial composition of claim 17 comprising
(i) benzyl alcohol at a concentration between about 0.3 and 1.0 percent (weight/weight);
(ii) Biosecur® or other citrus extract at a concentration between about 0.3 and 1.0 percent (weight/weight);
(iii) ethyl lauroyl arginate/glyceryl laurate at a concentration between about 0.02 and 0.05 percent (weight/weight);
(iv) an agent selected from the group consisting of thymol, thyme oil, and combinations thereof, at a concentration between about 0.02 and 0.1 percent (weight/weight); and
(v) organic acid selected from the group consisting of benzoic acid, lactic acid and combinations thereof at a concentration between about 0 and 0.2 percent (weight/weight).

22. The antimicrobial composition of claim 17 comprising
(i) benzyl alcohol at a concentration between about 5 and 30 percent (weight/weight);
(ii) Biosecur® or other citrus extract at a concentration between about 2 and 20 percent (weight/weight);
(iii) ethyl lauroyl arginate/glyceryl laurate at a concentration between about 1 and 5 percent (weight/weight);
(iv) an agent selected from the group consisting of thymol, thyme oil, and combinations thereof, at a concentration between about 0.1 and 0.4 percent (weight/weight);
(v) cinnamon oil at a concentration between about 2 and 10 percent (weight/weight), wherein the cinnamon oil comprises cinnamon bark oil;
(vi) solvent selected from the group consisting of ethanol, glycerine, and combinations thereof, at a concentration between about 5 and 70 percent (weight/weight); and
(vii) organic acid selected from the group consisting of benzoic acid, lactic acid and combinations thereof at a concentration between about 0 and 20 percent (weight/weight).

23. The antimicrobial composition of claim 17 comprising
(i) benzyl alcohol at a concentration between about 0.01 and 0.06 percent (weight/weight);
(ii) Biosecur® or other citrus extract at a concentration between about 0.004 and 0.04 percent (weight/weight);
(iii) ethyl lauroyl arginate/glyceryl laurate at a concentration between about 0.002 and 0.01 percent (weight/weight);
(iv) an agent selected from the group consisting of thymol, thyme oil, and combinations thereof, at a concentration between about 0.0002 and 0.0008 percent (weight/weight);
(v) cinnamon oil at a concentration between about 0.004 and 0.02 percent (weight/weight), wherein the cinnamon oil comprises cinnamon bark oil;
(vi) solvent selected from the group consisting of ethanol, glycerine, and combinations thereof, at a concentration between about 0.01 and 0.15 percent (weight/weight); and
(vii) organic acid selected from the group consisting of benzoic acid, lactic acid and combinations thereof at a concentration between about 0 and 0.2 percent (weight/weight).

24. The antimicrobial composition of claim 17 comprising
(i) benzyl alcohol at a concentration between about 0.5 and 1.0 percent (weight/weight);
(ii) Biosecur® or other citrus extract at a concentration between about 0.5 and 1.0 percent (weight/weight);
(iii) ethyl lauroyl arginate/glyceryl laurate at a concentration between about 0.001 and 0.05 percent (weight/weight);
(iv) an agent selected from the group consisting of thymol, thyme oil, and combinations thereof, at a concentration between about 0.001 and 0.5 percent (weight/weight); and
(v) organic acid selected from the group consisting of benzoic acid, lactic acid and combinations thereof at a concentration between about 0 and 0.2 percent (weight/weight).

25. The antimicrobial composition of claim 17 comprising
(i) benzyl alcohol at a concentration between about 0.1 and 0.5 percent (weight/weight);
(ii) Biosecur® or other citrus extract at a concentration between about 0.1 and 2.0 percent (weight/weight);
(iii) ethyl lauroyl arginate/glyceryl laurate at a concentration between about 0.02 and 0.2 percent (weight/weight);
(iv) an agent selected from the group consisting of thymol, thyme oil, and combinations thereof, at a concentration between about 0.03 and 0.1 percent (weight/weight);
(v) peppermint oil at a concentration between about 0.02 and 0.1 percent (weight/weight), or menthol at a concentration between about 0.01 and 0.1 percent (weight/weight), or a combination thereof, wherein the peppermint oil and/or menthol is a flavoring agent; and
(v) organic acid selected from the group consisting of benzoic acid, lactic acid and combinations thereof at a concentration between about 0 and 0.2 percent (weight/weight).

26. The antimicrobial composition of claim 17 comprising
(i) benzyl alcohol at a concentration between about 0.5 and 30 percent (weight/weight);
(ii) Biosecur® or other citrus extract at a concentration between about 5 and 40 percent (weight/weight);
(iii) ethyl lauroyl arginate/glyceryl laurate at a concentration between about 0.2 and 4 percent (weight/weight); and
(iv) organic acid selected from the group consisting of benzoic acid, lactic acid and combinations thereof at a concentration between about 0 and 20 percent (weight/weight).

27. The antimicrobial composition of claim 17 comprising
(i) benzyl alcohol at a concentration between about 0.1 and 1 percent (weight/weight);
(ii) Biosecur® or other citrus extract at a concentration between about 0.1 and 1 percent (weight/weight);
(iii) ethyl lauroyl arginate/glyceryl laurate at a concentration between about 0.05 and 0.5 percent (weight/weight); and
(iv) organic acid selected from the group consisting of benzoic acid, lactic acid and combinations thereof at a concentration between about 0 and 0.2 percent (weight/weight).

28. An antimicrobial composition comprising
(ii) Biosecur® or other citrus extract;
(ii) ethyl lauroyl arginate/glyceryl laurate;
(iii) thymol, thyme oil, or a combination thereof; and
(iv) organic acid selected from the group consisting of benzoic acid, lactic acid and combinations thereof.

29. The antimicrobial composition of claim 28 comprising
(i) Biosecur® or other citrus extract at a concentration between about 5 and 40 percent (weight/weight);
(ii) ethyl lauroyl arginate/glyceryl laurate at a concentration between about 0.2 and 4 percent (weight/weight);
(iii) thymol, thyme oil, or a combination thereof, at a concentration between about 0.1 and 5 percent (weight/weight); and
(iv) organic acid selected from the group consisting of benzoic acid, lactic acid and combinations thereof at a concentration between about 0 and 20 percent (weight/weight).

30. The antimicrobial composition of claim 28 comprising
(ii) Biosecur® or other citrus extract at a concentration between about 0.1 and 1 percent (weight/weight);
(ii) ethyl lauroyl arginate/glyceryl laurate at a concentration between about 0.02 and 0.5 percent (weight/weight);
(iii) thymol, thyme oil, or a combination thereof, at a concentration between about 0.01 and 0.5 percent (weight/weight); and
(iv) organic acid selected from the group consisting of benzoic acid, lactic acid and combinations thereof at a concentration between about 0 and 0.2 percent (weight/weight).

31. An antimicrobial composition comprising
(i) benzyl alcohol;
(ii) Biosecur® or other citrus extract;
(iii) one or more agent selected from the group consisting of thymol, thyme oil, peppermint oil, menthol, and organic acid selected from the group consisting of benzoic acid, lactic acid and combinations thereof.

32. The antimicrobial composition of claim 31 comprising
(i) benzyl alcohol at a concentration between about 0.1 and 0.5 percent (weight/weight);
(ii) Biosecur® or other citrus extract at a concentration between about 0.1 and 2.0 percent (weight/weight);
(iii) thymol, thyme oil, or a combination thereof, at a concentration between about 0.03 and 0.1 percent (weight/weight);
(iv) peppermint oil at a concentration between about 0.02 and 0.1 percent (weight/weight), or menthol at a concentration between about 0.01 and 0.1 percent (weight/weight), or a combination thereof, wherein the peppermint oil and/or menthol is a flavoring agent; and
(v) organic acid selected from the group consisting of benzoic acid, lactic acid and combinations thereof at a concentration between about 0 and 0.2 percent (weight/weight).

33. The antimicrobial composition of claim 31 comprising
(i) benzyl alcohol at a concentration between about 10 and 70 percent (weight/weight);
(ii) Biosecur® or other citrus extract at a concentration between about 1 and 80 percent (weight/weight);
(iii) thymol, thyme oil, or a combination thereof, at a concentration between about 0.5 and 20 percent (weight/weight); and
(iv) organic acid selected from the group consisting of benzoic acid, lactic acid and combinations thereof at a concentration between about 0 and 20 percent (weight/weight).

34. A method of reducing the number and/or inhibiting the growth of microbes in or on a product or inanimate surface comprising incorporating an antimicrobial composition according to claim 1 into the product or inanimate surface, or exposing the product or inanimate surface to an antimicrobial composition according to claim 1, wherein the product is selected from the group consisting of a personal care product, a food product, and a beverage product.

35. A method of reducing the number and/or inhibiting the growth of microbes on the skin or in the mouth of a subject comprising applying an antimicrobial composition according to claim 1 to the skin or an inside surface of the mouth of the subject.
